(19) United States
(12) Patent Application Publication
(10) Pub. No.: US 2024/0156860 A1
(43) Pub. Date: May 16, 2024

(54) METHOD OF TREATING INFECTED AND NONINFECTED BURN WOUNDS

(71) Applicant: UNIVERSITY OF CINCINNATI, CINCINNATI, OH (US)

(72) Inventors: DANIEL HASSETT, CINCINNATI, OH (US); LATHA SATISH, MASON, OH (US)

(21) Appl. No.: 18/282,627

(22) PCT Filed: Mar. 17, 2022
(86) PCT No.: PCT/US2022/020751

§ 371 Date: Sep. 18, 2023

Related U.S. Application Data
(60) Provisional application No. 63/162,670, filed on Mar. 18, 2021.

(51) Int. Cl. A61K 33/00; A61K 9/00; A61K 9/06; A61K 31/198; A61K 47/10; A61P 17/02; A61P 31/04

(52) U.S. Cl. CPC A61K 33/00; A61K 9/0014; A61K 9/06; A61K 31/198; A61K 47/10; A61P 17/02; A61P 31/04

(57) ABSTRACT

A method for treating a subject having a cutaneous thermal injury is disclosed. The method comprises administering a therapeutically effective amount of a bactericidal compound topically to the injury. Use of a therapeutically effective amount of a bactericidal compound topically for treating a subject having a cutaneous thermal injury is also disclosed. The bactericidal compound comprises a therapeutically effective amount of NaNO₂ (A-NO₂—) and di-sodium EDTA (Na₂-EDTA).

### RELATED APPLICATION

This disclosure claims benefit of U.S. Provisional Patent Application Ser. No. 63/162,670 filed Mar. 18, 2021, incorporated herein by reference in its entirety.

### TECHNICAL FIELD

The present disclosure relates to a method of treating infected and noninfected burn wounds. In some embodiments, it relates to a method of treating bacterial burn wound infections.

### BACKGROUND

Cutaneous thermal injuries (i.e. burn or blast injuries) are a major cause of morbidity and mortality. This is largely due to sepsis, the most expensive health-care problem in the U.S., which costs >$20 billion a year. Sepsis is often preceded by wound infection, which triggers delayed wound healing. The major challenge in treating bacterial infections is their inherent or acquired antibiotic resistance. Pseudomonas aeruginosa (PA) is a multi-drug resistant (MDR) Gram-negative (G−) bacterium, responsible for over half of all severe burn infections and identified as a “major pathogen” by the Centers for Disease Control and Prevention (CDC). Due to the intrinsically acquired resistance of PA to many conventional antimicrobial regimens, treatment strategies for burn wound infections caused by PA are both challenging and limited. Hence, there is a critical need to develop novel and effective antimicrobials for the (i) prevention, (ii) treatment and (iii) healing of burn/blast wounds that are complicated by bacterial infections.

### SUMMARY

The present disclosure relates to a method for treating a subject having a cutaneous thermal injury. The method comprises administering a therapeutically effective amount of a bactericidal compound topically to the injury, wherein the bactericidal compound comprises a therapeutically effective amount of acidified NaNO₂ (A-NO₂⁻) and di-sodium EDTA (Na₂-EDTA).

In one embodiment, a preventative effective amount of the bactericidal compound is administered. In another embodiment, the bactericidal compound is in a composition that further comprises a water-based gel.

In one embodiment, the water-based gel comprises glycerol, an absorbent polymer and water. In another embodiment, the water-based gel comprises glycerol in the range from about 10 to about 30 percent by volume. In another embodiment, the bactericidal compound has a pH in the range from about 6.0 to about 6.5.

In one embodiment, the cutaneous thermal injury comprises a bacterial infection. In another embodiment, the bacterial infection is caused by a multi-drug resistant (MDR) gram-negative bacteria. In another embodiment, the MDR gram-negative bacteria comprises Pseudomonas aeruginosa.

In one embodiment, the bactericidal compound comprises from about 1 to about 5 mM of Na₂-EDTA and from about 20 to about 40 mM of NaNO₂. In another embodiment, the bactericidal compound comprises about 2 mM of di-sodium EDTA and about 30 mM of NaNO₂. In another embodiment, the bactericidal compound comprises from about 20 to about 40 mM of Na₂-EDTA and from about 400 to about 600 mM NaNO₂. In another embodiment, the bactericidal compound comprises about 33 mM of Na₂-EDTA and about 500 mM NaNO₂.

In one embodiment, the present disclosure relates to a composition including a therapeutically effective amount of acidified NaNO₂ (A-NO₂⁻) and Na₂-EDTA and a water-based gel. In another embodiment, the water-based gel comprises glycerol, an absorbent polymer and water. In another embodiment, the water-based gel comprises glycerol in the range from about 10 to about 30 percent by volume. In another embodiment, the bactericidal compound has a pH in the range from about 6.0 to about 6.5.

In one embodiment, the bactericidal compound comprises from about 1 to about 5 mM of Na₂-EDTA and from about 20 to about 40 mM of NaNO₂. In another embodiment, the bactericidal compound comprises from about 20 to about 40 mM of Na₂-EDTA and from about 400 to about 600 mM NaNO₂. In another embodiment, the bactericidal compound has a ratio of acidified NaNO₂ (A-NO₂—) and Na₂-EDTA to water-based gel of about 1:100 (w/w).

In another aspect, the present disclosure provides a use of a therapeutically effective amount of a bactericidal compound for treating a subject having a cutaneous thermal injury. In one embodiment, the bactericidal compound comprises a therapeutically effective amount of acidified NaNO₂ (A-NO₂⁻) and di-sodium EDTA (Na₂-EDTA).

In another aspect, the present disclosure provides a use of a therapeutically effective amount of a bactericidal compound for the manufacture of a medicament for treating a subject having a cutaneous thermal injury. In one embodiment, the bactericidal compound comprises a therapeutically effective amount of acidified NaNO₂ (A-NO₂⁻) and di-sodium EDTA (Na₂-EDTA).

In another aspect, the present disclosure provides a therapeutically effective amount of a bactericidal compound for use in treating a subject having a cutaneous thermal injury. In one embodiment, the bactericidal compound comprises a therapeutically effective amount of acidified NaNO₂ (A-NO₂⁻) and di-sodium EDTA (Na₂-EDTA).

Also provided is a method for enhancing wound healing in a subject having a cutaneous thermal wound. In one embodiment, the method comprises administering a therapeutically effective amount of a bactericidal compound topically to the wound. In one embodiment, the bactericidal compound comprises a therapeutically effective amount of acidified NaNO₂ (A-NO₂—) and Na₂-EDTA.

In another aspect, also provided is a topical use of a therapeutically effective amount of a bactericidal compound for enhancing wound healing in a subject having a cutaneous thermal wound. In one embodiment, the bactericidal compound comprises a therapeutically effective amount of acidified NaNO₂ (A-NO₂—) and Na₂-EDTA.

In another aspect, also provided is a use of a therapeutically effective amount of a bactericidal compound in the manufacture of a topical medicament for enhancing wound healing in a subject having a cutaneous thermal wound. In one embodiment, the bactericidal compound comprises a therapeutically effective amount of acidified NaNO₂ (A-NO₂—) and Na₂-EDTA.

In another aspect, also provided is a therapeutically effective amount of a bactericidal compound for topical use in enhancing wound healing in a subject having a cutaneous thermal wound. In one embodiment, the bactericidal compound comprises a therapeutically effective amount of acidified NaNO₂ (A-NO₂—) and Na₂-EDTA.

Other features and advantages of the present disclosure will become apparent from the following detailed description. It should be understood, however, that the detailed description, while indicating preferred implementations of the present disclosure, is given by way of illustration only, since various changes and modification within the spirit and scope of the disclosure will become apparent to those of skill in the art from the detailed description.

### BRIEF DESCRIPTION OF THE DRAWINGS

FIG. 1A is a graph showing the results of a broth based killing assay. It shows that PA strain XEN41 killing is complete and maintained 48 hours after treatment with 30 mM A-NO₂⁻ (acidified nitrite) and 2 mM Na₂-EDTA and 30 mM A-NO₂⁻.

FIG. 1B is an illustration of representative IVIS images of XEN41-containing plates showing elimination of bioluminescent signals of this PAO1-derivative using A-NO₂—and AB569 48 hours after treatment.

FIG. 1C is a graph showing that AB569 gel exhibited significant anti-bacterial activity on PA clinical strains (N=8) isolated from burn wounds irrespective of their response to various antibiotics. Strains 1-3 were either sensitive or showed an intermediate response to various antibiotics; strains 4 & 7 were resistant to various antibiotics but was sensitive to AB569; strains 5, 6 & 9 were sensitive to various antibiotics and to AB569.

FIG. 2 is a checkerboard assay of clinical strains of PA isolated from burn wounds (N=21) and strain PAO1 subjected to AB569 gel sensitivity testing.

FIG. 3A is an image of nitric oxide (NO) measurements performed using an ISO—NOP probe linked to an Apollo 4000 detector. pA-unit was used to detect NO levels. The figure shows amperometric detection of NO release from the AB569-gel formulation.

FIG. 3B is an image of nitric oxide (NO) measurements done using an ISO—NOP probe linked to an Apollo 4000 detector. The figure shows amperometric detection of NO release without AB569.

FIG. 4A is a series of representative IVIS images of burn wounds infected and treated with high (33 mM Na₂-EDTA+500 mM A-NO₂⁻ formulated in 1% SS gel) and low dose of AB569 (2 mM Na₂-EDTA+30 mM A-NO₂⁻ in 1% SS gel) shown on PBD 1, 2, 3, 6, 8, and 16.

FIG. 4B is a graph of photon intensities of bacterial burden showing significant reduction in photon intensities of PAO1 XEN41 in animals treated with low and high AB569.

FIG. 5A is a series of images showing gross images of the wounds, and analysis of wound closure was performed using NIH Image J. Wounds were photographed weekly using a standard digital camera.

FIG. 5B is a graph showing rate of wound closure in uninfected treated burn wounds measured by Image J from burn day to post-burn day (PBD) 29.

FIG. 5C is a series of images showing gross images of the wounds, and analysis of wound closure was performed using NIH Image J. Wounds were photographed weekly using a standard digital camera.

FIG. 5D is a graph showing rate of wound closure in uninfected treated burn wounds measured by Image J from burn day to PBD 29.

FIG. 6A is a graph showing the survival rates of uninfected burn wounds from Burn day to PBD 29 in all the treatment groups (Total no. of animals; n=87).

FIG. 6B is a graph showing the survival rates of infected wounds from Burn day to PBD 24 in all the treatment groups (Total no. of animals; n=109).

FIG. 7A is a graph showing body weight for the uninfected burn wounds and different treatments over time.

FIG. 7B is a graph showing body weight for the infected burn wounds and different treatments over time.

FIG. 8A is a graph showing spleen weights for the uninfected burn wounds and various treatments.

FIG. 8B is a graph showing spleen weight for the infected burn wounds and treatments.

FIG. 8C is a graph showing body to spleen ratio for the uninfected burn wounds and treatments.

FIG. 8D is a graph showing body to spleen ratio for the infected burn wounds and treatments.

FIG. 9A is a graph showing fold change expression of RNA isolated from treated and untreated burn wound tissue from PBD 29 on IL-6 and IL-10 compared to burn alone.

FIG. 9B is a graph showing serum cytokine levels of IL-6 samples.

FIG. 9C is a graph showing serum cytokine levels of IL-10 samples.

FIG. 9D is a graph showing serum cytokine levels of IL-10 samples.

FIG. 9E is a graph showing serum cytokine levels of G-CSF samples.

FIG. 10A is a series of images showing gross images of H&E stained sections which were untreated or treated with SS, L-AB569, H-AB569 or PAO1+H-AB569.

FIG. 10B is a graph showing fold change expression of RNA isolated from treated and untreated burn wound tissue on type I collagen (COL1A1) and type III collagen (COL3A1) compared to burn alone.

FIG. 10C is a series of images showing gross images of sections stained with Masson's trichrome stain which were untreated or treated with SS, L-AB569, H-AB569 or PAO1+H-AB569.

FIG. 11 is a series of images showing burn wound sections from control and various treatment groups harvested on PBD 29 stained with Ki67.

FIG. 12A is a graph showing efficient rRNA depletion from mixed mouse and P. aeruginosa total RNA. The figure shows total RNA extracted from the infected tissue, which showed both mouse 18S and 28S rRNA (peak 2 and 4) and P. aeruginosa 16S and 23S rRNA (peak 1 and 3).

FIG. 12B is a graph showing that after rRNA depletion, all 4 rRNA peaks disappeared, indicating efficient rRNA depletion for dual RNA-seq.

### DETAILED DESCRIPTION

### Definitions

All technical and scientific terms used herein, unless otherwise defined below, are intended to have the same meaning as commonly understood by one of ordinary skill in the art. References to methods employed herein are intended to refer to the methods as commonly understood in the art, including variations on those methods or substitutions of equivalent methods that would be apparent to one of skill in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

As used in this specification, the singular forms “a”, “an” and “the” specifically also encompass the plural forms of the terms to which they refer, unless the content clearly dictates otherwise. For example, reference to “antimicrobial” includes mixtures of antimicrobials.

The term “infection” as used herein means and/or colonization by a microorganism and/or multiplication of a micro-organism, in particular, a bacterium. The bacterium can be Gram-negative such as the Pseudomonas genus (e.g., species aeruginosa), or Gram-positive (e.g., Staphylococcus aureus) in a subject. Such infection may be unapparent or result in local cellular injury. The infection may be localized, subclinical and temporary or alternatively may spread by extension to become an acute or chronic clinical infection. The infection may also be a past infection wherein residual antigen from a protein associated with aerobic, microaerobic or anaerobic growth of P. aeruginosa, or alternatively, reactive host antibodies that bind to isolated from a protein of P. aeruginosa protein or peptides there from, remain in the host. The infection may also be a latent infection, in which the microorganism is present in a subject, however the subject does not exhibit symptoms of disease associated with the organism. The infection can be a respiratory infection by P. aeruginosa, i.e., an infection of the respiratory tract. The term infection also encompasses a P. aeruginosa infection of a wound (e.g., a burn/blast/diabetic), an infection of the meninges (e.g., meningitis), a urinary tract infection, an infection of a heart valve (e.g., endocarditis), an ear infection, an eye infection, a bone infection (e.g., Vertebral osteomyelitis), a skin infection or a gastro-intestinal infection.

The term “absorbent polymer” as used herein means a hydrophilic or amphiphilic polymeric network composed of homopolymers or copolymers, which is insoluble due to the presence of covalent chemical crosslinks. The crosslinks provide the network structure and physical integrity.

The term “subject” or “individual” or “patient” is meant to include any subject, particularly a mammalian subject, including human, for whom diagnosis, prognosis, or therapy is desired. The subject or patient is suitably a human.

The term a “therapeutically effective amount” as used herein means an amount of a compound or a composition, which when administered according to a desired dosage regimen, is sufficient to at least partially attain the desired therapeutic effect, or delay the onset of, or inhibit the progression of, halt, partially or fully the onset or progression of the infection or is able to reverse or partially reverse the antimicrobial sensitivity of the pathogenic microbe(s), or enhance wound healing.

The term a “preventative effective amount” as used herein means an amount of a compound or a composition, which when administered according to a desired dosage regimen, is sufficient to at least partially prevent or delay the onset of the infection.

As used herein, “treating” or “treatment” refers to inhibiting the disease or condition, i.e., arresting or reducing its development or at least one clinical or subclinical symptom thereof. “Treating” or “treatment” further refers to relieving the disease or condition, i.e., causing regression of the disease or condition or at least one of its clinical or subclinical symptoms. The benefit to a patient to be treated is either statistically significant or at least perceptible to the patient and/or the physician. In the context of treating a bacterial infection, the term treatment includes reducing or eliminating colonization by a bacteria and/or multiplication of a bacteria including reducing biofilm formation or disrupting existing biofilms.

As used herein, the term “administering” refers to a method of giving a dosage of a pharmaceutical composition of the disclosure to a subject. The compositions utilized in the methods described herein can be administered by a route selected from, e.g., parenteral, dermal, transdermal, ocular, inhalation, buccal, sublingual, perilingual, nasal, rectal, topical administration, intravesicular and oral administration. Specific administration methods are described in further detail herein. Parenteral administration includes intravenous, intraperitoneal, subcutaneous, intraarterial, intravascular, and intramuscular administration. The preferred method of administration can vary depending on various factors (e.g., the components of the composition being administered and the severity of the condition being treated).

The recitation of numerical ranges by endpoints herein includes all numbers and fractions subsumed within that range (e.g. 1 to 5 includes for example 1, 1.5, 2, 2.75, 3, 3.90, 4, and 5). It is also to be understood that all numbers and fractions thereof are presumed to be modified by the term “about”.

### Treatment for Burn Wound and Bactericidal Compound

The present disclosure is directed toward novel methods and uses for treatment of burn wounds.

Despite improvements in early treatment, survival following burn injury remains challenging, due largely to sepsis, the leading cause of death in pediatric and adult burn patients. Sepsis is often preceded by infectious complications. One of the greatest challenges in treating bacterial infections is their resistance to conventional antibiotic. Multi-drug resistant (MDR) bacteria now account for the bulk of deaths due to sepsis, which is the most expensive health-care problem in the United States (U.S.), with a cost of more than $20 billion annually. Infection is an even greater cause of death from burn trauma in military personnel than in the general population. Pseudomonas aeruginosa (PA) is the most frequently cultured source of infection in burn patients, accounting for over half of all severe burn infections, and is among the major cause of sepsis after burn trauma. Within a few days of admission, 14-33% of burn wounds are colonized with PA. Moreover, infection is the main cause of delayed wound healing in various types of wounds, including burns. The Centers for Disease Control and Prevention (CDC) has earmarked PA as a major pathogen and MDR organism responsible for life-threatening infections in critically ill or immune-compromised patients. Due to the intrinsically high acquired antibiotic resistance of PA to many, and in some cases, all of the conventional antimicrobial treatments used to date, treatment of burn wound infections caused by PA is both challenging and frustratingly limited, especially since the development of promising new antimicrobial agents has slowed to a trickle. Hence, there is a critical and urgent unmet need to develop novel and effective antimicrobials for the treatment and prevention of bacterial burn/blast/wound infections by formidable pathogens such as MDR-PA.

AB569, an innovative, bactericidal combination of acidified nitrite (A-NO₂—) and Na₂-EDTA, has broad-spectrum activity against virtually all pathogenic bacteria. Regarding human use, the NaNO₂ and/or Na₂-EDTA component(s) of AB569 have separately been proven safe in studies related to the treatment of cyanide poisoning, burn wounds, cystic fibrosis (CF) lung infection, urinary tract infection, wound healing, chelation therapy, and cosmetics. Furthermore, both components of AB569 have been reported to increase the efficacy of certain antibiotics that are commonly used to treat a variety of infections. AB569 has excellent bactericidal activity against all tested Gram-positive (G+) and Gram-negative (G−) bacteria including those that are MDR. Importantly, there was no observed discernable toxicity of AB569 to human airway (e.g., CF), skin (e.g., burn wounds) or bladder (e.g., UTI's) cells or in a mouse model of PA airway infection and no development of resistance by bacteria cultured in vitro. However, little is known regarding the potential of the A-NO₂— and Na₂-EDTA combination in the treatment of PA-mediated burn wound infection and in wound healing, a far more clinically simpler topical assessment of AB569 efficacy than complicated airway delivery systems.

The present disclosure uses AB569 to reduce the PA burden in burn wounds, and to promote wound closure and scar reduction. Further, the present disclosure uses AB569 to enhance wound contraction and heal uninfected burn wounds with reduced scar formation. The inventors have developed a water-based gel formulation of AB569, which can easily be applied topically to wounds, and tested its efficacy on clinical strains of PA isolated from human burn patients. Strikingly, bacterial killing was observed in all PA strains tested. Furthermore, the inventors have successfully established a burn wound infection mouse model that presents overt signs of infection following PA inoculation, which allows the inventors to determine the in vivo effectiveness of AB569 application in a complex, infected wound/burn niche. Identification of transcriptomic changes in burn wound-related PA upon treatment with AB569 provides mechanistic insight into its bactericidal mode of action.

Accordingly, the present disclosure provides a method for treating a subject having a cutaneous thermal injury. In one embodiment, the method comprises administering a therapeutically effective amount of a bactericidal compound topically to the injury. In one embodiment, the bactericidal compound comprises a therapeutically effective amount of acidified NaNO₂ (A-NO₂⁻) and di-sodium EDTA (Na₂-EDTA). In one embodiment, a preventative effective amount of the bactericidal compound is administered. In another embodiment, the bactericidal compound is in a composition that further comprises a water-based gel. In one embodiment, the water-based gel comprises glycerol, an absorbent polymer and water. In another embodiment, the water-based gel comprises glycerol in the range from about 10 to about 30 percent by volume. In another embodiment, the water-based gel comprises an absorbent polymer in the range from about 1 to about 10 percent by volume. In another embodiment, the bactericidal compound has a pH in the range from about 6.0 to about 6.5. In one embodiment, the cutaneous thermal injury comprises a bacterial infection. In one embodiment, the cutaneous thermal injury comprises a noninfected burn wound. In another embodiment, the bacterial infection comprises a multi-drug resistant (MDR) gram-negative bacterial infection. In another embodiment, the bacterial infection is a MDR gram-negative bacterial infection. In another embodiment, the MDR gram-negative bacteria comprises Pseudomonas aeruginosa. In another embodiment, the MDR gram-negative bacterial infection is caused by Pseudomonas aeruginosa. In one embodiment, the bactericidal compound comprises from about 1 to about 5 mM of Na₂-EDTA and from about 20 to about 40 mM of NaNO₂. In another embodiment, the bactericidal compound comprises about 2 mM of di-sodium EDTA (Na₂-EDTA) and about 30 mM of NaNO₂. In another embodiment, the bactericidal compound comprises from about 20 to about 40 mM of Na₂-EDTA and from about 400 to about 600 mM NaNO₂. In another embodiment, the bactericidal compound comprises about 33 mM of Na₂-EDTA and about 500 mM NaNO₂. In one embodiment, the NaNO₂ is acidified NaNO₂. In another embodiment, the bactericidal compound has a ratio of acidified NaNO₂ (A-NO₂—) and Na₂-EDTA to water-based gel of about 1:100 (w/w). In one embodiment, the bactericidal compound comprises AB569. In another embodiment, the bactericidal compound is in a composition comprising AB569 and a water-based gel. In another embodiment, the water-based gel comprises Solosite®. In one embodiment, the subject is a mammal. In one embodiment, the subject is a human.

In another aspect, the present disclosure provides a use of a therapeutically effective amount of a bactericidal compound for treating a subject having a cutaneous thermal injury. In one embodiment, the bactericidal compound comprises a therapeutically effective amount of acidified NaNO₂ (A-NO₂⁻) and di-sodium EDTA (Na₂-EDTA). In one embodiment, the bactericidal compound comprises a preventative effective amount of the bactericidal compound. In another embodiment, the bactericidal compound is in a composition that further comprises a water-based gel. In one embodiment, the water-based gel comprises glycerol, an absorbent polymer and water. In another embodiment, the water-based gel comprises glycerol in the range from about 10 to about 30 percent by volume. In another embodiment, the water-based gel comprises an absorbent polymer in the range from about 1 to about 10 percent by volume. In another embodiment, the bactericidal compound has a pH in the range from about 6.0 to about 6.5. In one embodiment, the cutaneous thermal injury comprises a bacterial infection. In one embodiment, the cutaneous thermal injury comprises a noninfected burn wound. In another embodiment, the bacterial infection comprises a multi-drug resistant (MDR) gram-negative bacterial infection. In another embodiment, the bacterial infection is a MDR gram-negative bacterial infection. In another embodiment, the MDR gram-negative bacteria comprises Pseudomonas aeruginosa. In another embodiment, the MDR gram-negative bacterial infection is caused by Pseudomonas aeruginosa. In one embodiment, the bactericidal compound comprises from about 1 to about 5 mM of Na₂-EDTA and from about 20 to about 40 mM of NaNO₂. In another embodiment, the bactericidal compound comprises about 2 mM of di-sodium EDTA (Na₂-EDTA) and about 30 mM of NaNO₂. In another embodiment, the bactericidal compound comprises from about 20 to about 40 mM of Na₂-EDTA and from about 400 to about 600 mM NaNO₂. In another embodiment, the bactericidal compound comprises about 33 mM of Na₂-EDTA and about 500 mM NaNO₂. In one embodiment, the NaNO₂ is acidified NaNO₂. In another embodiment, the bactericidal compound has a ratio of acidified NaNO₂ (A-NO₂—) and Na₂-EDTA to water-based gel of about 1:100 (w/w). In one embodiment, the bactericidal compound comprises AB569. In another embodiment, the bactericidal compound is in a composition comprising AB569 and a water-based gel. In another embodiment, the water-based gel comprises Solosite®. In one embodiment, the subject is a mammal. In one embodiment, the subject is a human.

In another aspect, the present disclosure provides a use of a therapeutically effective amount of a bactericidal compound for the manufacture of a medicament for treating a subject having a cutaneous thermal injury. In one embodiment, the bactericidal compound comprises a therapeutically effective amount of acidified NaNO₂ (A-NO₂⁻) and di-sodium EDTA (Na₂-EDTA). In one embodiment, the bactericidal compound comprises a preventative effective amount of the bactericidal compound. In another embodiment, the bactericidal compound is in a composition that further comprises a water-based gel. In one embodiment, the water-based gel comprises glycerol, an absorbent polymer and water. In another embodiment, the water-based gel comprises glycerol in the range from about 10 to about 30 percent by volume. In another embodiment, the water-based gel comprises an absorbent polymer in the range from about 1 to about 10 percent by volume. In another embodiment, the bactericidal compound has a pH in the range from about 6.0 to about 6.5. In one embodiment, the cutaneous thermal injury comprises a bacterial infection. In one embodiment, the cutaneous thermal injury comprises a noninfected burn wound. In another embodiment, the bacterial infection comprises a multi-drug resistant (MDR) gram-negative bacterial infection. In another embodiment, the bacterial infection is a MDR gram-negative bacterial infection. In another embodiment, the MDR gram-negative bacteria comprises Pseudomonas aeruginosa. In another embodiment, the MDR gram-negative bacterial infection is caused by Pseudomonas aeruginosa. In one embodiment, the bactericidal compound comprises from about 1 to about 5 mM of Na₂-EDTA and from about 20 to about 40 mM of NaNO₂. In another embodiment, the bactericidal compound comprises about 2 mM of di-sodium EDTA (Na₂-EDTA) and about 30 mM of NaNO₂. In another embodiment, the bactericidal compound comprises from about 20 to about 40 mM of Na₂-EDTA and from about 400 to about 600 mM NaNO₂. In another embodiment, the bactericidal compound comprises about 33 mM of Na₂-EDTA and about 500 mM NaNO₂. In one embodiment, the NaNO₂ is acidified NaNO₂. In one embodiment, the subject is a mammal. In one embodiment, the subject is a human. ###

In another aspect, the present disclosure provides a therapeutically effective amount of a bactericidal compound for use in treating a subject having a cutaneous thermal injury. In one embodiment, the bactericidal compound comprises a therapeutically effective amount of acidified NaNO₂ (A-NO₂⁻) and di-sodium EDTA (Na₂-EDTA). In one embodiment, the bactericidal compound comprises a preventative effective amount of the bactericidal compound. In another embodiment, the bactericidal compound is in a composition that further comprises a water-based gel. In one embodiment, the water-based gel comprises glycerol, an absorbent polymer and water. In another embodiment, the water-based gel comprises glycerol in the range from about 10 to about 30 percent by volume. In another embodiment, the water-based gel comprises an absorbent polymer in the range from about 1 to about 10 percent by volume. In another embodiment, the bactericidal compound has a pH in the range from about 6.0 to about 6.5. In one embodiment, the cutaneous thermal injury comprises a bacterial infection. In one embodiment, the cutaneous thermal injury comprises a noninfected burn wound. In another embodiment, the bacterial infection comprises a multi-drug resistant (MDR) gram-negative bacterial infection. In another embodiment, the bacterial infection is a MDR gram-negative bacterial infection. In another embodiment, the MDR gram-negative bacteria comprises Pseudomonas aeruginosa. In another embodiment, the MDR gram-negative bacterial infection is caused by Pseudomonas aeruginosa. In one embodiment, the bactericidal compound comprises from about 1 to about 5 mM of Na₂-EDTA and from about 20 to about 40 mM of NaNO₂. In another embodiment, the bactericidal compound comprises about 2 mM of di-sodium EDTA (Na₂-EDTA) and about 30 mM of NaNO₂. In another embodiment, the bactericidal compound comprises from about 20 to about 40 mM of Na₂-EDTA and from about 400 to about 600 mM NaNO₂. In another embodiment, the bactericidal compound comprises about 33 mM of Na₂-EDTA and about 500 mM NaNO₂. In one embodiment, the NaNO₂ is acidified NaNO₂. In another embodiment, the bactericidal compound has a ratio of acidified NaNO₂ (A-NO₂—) and Na₂-EDTA to water-based gel of about 1:100 (w/w). In one embodiment, the bactericidal compound comprises AB569. In another embodiment, the bactericidal compound is in a composition comprising AB569 and a water-based gel. In another embodiment, the water-based gel comprises Solosite®. In one embodiment, the subject is a mammal. In one embodiment, the subject is a human.

Also provided is a method for enhancing wound healing in a subject having a cutaneous thermal wound. In one embodiment, the method comprises administering a therapeutically effective amount of a bactericidal compound topically to the wound. In one embodiment, the bactericidal compound comprises a therapeutically effective amount of acidified NaNO₂ (A-NO₂—) and Na₂-EDTA. In one embodiment, a preventative effective amount of the bactericidal compound is administered. In another embodiment, the bactericidal compound is in a composition that further comprises a water-based gel. In one embodiment, the water-based gel comprises glycerol, an absorbent polymer and water. In another embodiment, the water-based gel comprises glycerol in the range from about 10 to about 30 percent by volume. In another embodiment, the water-based gel comprises an absorbent polymer in the range from about 1 to about 10 percent by volume. In another embodiment, the bactericidal compound has a pH in the range from about 6.0 to about 6.5. In one embodiment, the cutaneous thermal wound comprises a bacterial infection. In one embodiment, the cutaneous thermal wound has a bacterial infection. In one embodiment, the cutaneous thermal wound comprises a noninfected burn wound. In another embodiment, the bacterial infection comprises a multi-drug resistant (MDR) gram-negative bacterial infection. In another embodiment, the bacterial infection is a MDR gram-negative bacterial infection. In another embodiment, the MDR gram-negative bacteria comprises Pseudomonas aeruginosa. In another embodiment, the MDR gram-negative bacterial infection is caused by Pseudomonas aeruginosa. In one embodiment, the bactericidal compound comprises from about 1 to about 5 mM of Na₂-EDTA and from about 20 to about 40 mM of NaNO₂. In another embodiment, the bactericidal compound comprises about 2 mM of di-sodium EDTA (Na₂-EDTA) and about 30 mM of NaNO₂. In another embodiment, the bactericidal compound comprises from about 20 to about 40 mM of Na₂-EDTA and from about 400 to about 600 mM NaNO₂. In another embodiment, the bactericidal compound comprises about 33 mM of Na₂-EDTA and about 500 mM NaNO₂. In one embodiment, the NaNO₂ is acidified NaNO₂. In another embodiment, the bactericidal compound has a ratio of acidified NaNO₂ (A-NO₂—) and Na₂-EDTA to water-based gel of about 1:100 (w/w). In one embodiment, the bactericidal compound comprises AB569. In another embodiment, the bactericidal compound is in a composition comprising AB569 and a water-based gel. In another embodiment, the water-based gel comprises Solosite®. In one embodiment, the subject is a mammal. In one embodiment, the subject is a human.

In another aspect, the present disclosure provides a use of a therapeutically effective amount of a bactericidal compound for enhancing wound healing in a subject having a cutaneous thermal wound. In one embodiment, the bacterial compound is formulated for topical use. In one embodiment, the bactericidal compound comprises a therapeutically effective amount of acidified NaNO₂ (A-NO₂—) and Na₂-EDTA. In one embodiment, the bactericidal compound comprises a preventative effective amount of the bactericidal compound. In another embodiment, the bactericidal compound is in a composition that further comprises a water-based gel. In one embodiment, the water-based gel comprises glycerol, an absorbent polymer and water. In another embodiment, the water-based gel comprises glycerol in the range from about 10 to about 30 percent by volume. In another embodiment, the water-based gel comprises an absorbent polymer in the range from about 1 to about 10 percent by volume. In another embodiment, the bactericidal compound has a pH in the range from about 6.0 to about 6.5. In one embodiment, the cutaneous thermal wound comprises a bacterial infection. In one embodiment, the cutaneous thermal wound has a bacterial infection. In one embodiment, the cutaneous thermal wound comprises a noninfected burn wound. In another embodiment, the bacterial infection comprises a multi-drug resistant (MDR) gram-negative bacterial infection. In another embodiment, the bacterial infection is a MDR gram-negative bacterial infection. In another embodiment, the MDR gram-negative bacteria comprises Pseudomonas aeruginosa. In another embodiment, the MDR gram-negative bacterial infection is caused by Pseudomonas aeruginosa. In one embodiment, the bactericidal compound comprises from about 1 to about 5 mM of Na₂-EDTA and from about 20 to about 40 mM of NaNO₂. In another embodiment, the bactericidal compound comprises about 2 mM of di-sodium EDTA (Na₂-EDTA) and about 30 mM of NaNO₂. In another embodiment, the bactericidal compound comprises from about 20 to about 40 mM of Na₂-EDTA and from about 400 to about 600 mM NaNO₂. In another embodiment, the bactericidal compound comprises about 33 mM of Na₂-EDTA and about 500 mM NaNO₂. In one embodiment, the NaNO₂ is acidified NaNO₂. In another embodiment, the bactericidal compound has a ratio of acidified NaNO₂ (A-NO₂—) and Na₂-EDTA to water-based gel of about 1:100 (w/w). In one embodiment, the bactericidal compound comprises AB569. In another embodiment, the bactericidal compound is in a composition comprising AB569 and a water-based gel. In another embodiment, the water-based gel comprises Solosite®. In one embodiment, the subject is a mammal. In one embodiment, the subject is a human.

In another aspect, the present disclosure also provides a use of a therapeutically effective amount of a bactericidal compound for the manufacture of a medicament for enhancing wound healing in a subject having a cutaneous thermal wound. In one embodiment, the bacterial compound is formulated for topical use. In one embodiment, the bactericidal compound comprises a therapeutically effective amount of acidified NaNO₂ (A-NO₂—) and Na₂-EDTA. In one embodiment, the bactericidal compound comprises a preventative effective amount of the bactericidal compound. In another embodiment, the bactericidal compound is in a composition that further comprises a water-based gel. In one embodiment, the water-based gel comprises glycerol, an absorbent polymer and water. In another embodiment, the water-based gel comprises glycerol in the range from about 10 to about 30 percent by volume. In another embodiment, the water-based gel comprises an absorbent polymer in the range from about 1 to about 10 percent by volume. In another embodiment, the bactericidal compound has a pH in the range from about 6.0 to about 6.5. In one embodiment, the cutaneous thermal wound comprises a bacterial infection. In one embodiment, the cutaneous thermal wound has a bacterial infection. In one embodiment, the cutaneous thermal wound comprises a noninfected burn wound. In another embodiment, the bacterial infection comprises a multi-drug resistant (MDR) gram-negative bacterial infection. In another embodiment, the bacterial infection is a MDR gram-negative bacterial infection. In another embodiment, the MDR gram-negative bacteria comprises Pseudomonas aeruginosa. In another embodiment, the MDR gram-negative bacterial infection is caused by Pseudomonas aeruginosa. In one embodiment, the bactericidal compound comprises from about 1 to about 5 mM of Na₂-EDTA and from about 20 to about 40 mM of NaNO₂. In another embodiment, the bactericidal compound comprises about 2 mM of di-sodium EDTA (Na₂-EDTA) and about 30 mM of NaNO₂. In another embodiment, the bactericidal compound comprises from about 20 to about 40 mM of Na₂-EDTA and from about 400 to about 600 mM NaNO₂. In another embodiment, the bactericidal compound comprises about 33 mM of Na₂-EDTA and about 500 mM NaNO₂. In one embodiment, the NaNO₂ is acidified NaNO₂. In another embodiment, the bactericidal compound has a ratio of acidified NaNO₂ (A-NO₂—) and Na₂-EDTA to water-based gel of about 1:100 (w/w). In one embodiment, the bactericidal compound comprises AB569. In another embodiment, the bactericidal compound is in a composition comprising AB569 and a water-based gel. In another embodiment, the water-based gel comprises Solosite®. In one embodiment, the subject is a mammal. In one embodiment, the subject is a human.

Also provided is a therapeutically effective amount of a bactericidal compound for use in enhancing wound healing in a subject having a cutaneous thermal wound. In one embodiment, the bacterial compound is formulated for topical use. In one embodiment, the bactericidal compound comprises a therapeutically effective amount of acidified NaNO₂ (A-NO₂—) and Na₂-EDTA. In one embodiment, the bactericidal compound comprises a preventative effective amount of the bactericidal compound. In another embodiment, the bactericidal compound is in a composition that further comprises a water-based gel. In one embodiment, the water-based gel comprises glycerol, an absorbent polymer and water. In another embodiment, the water-based gel comprises glycerol in the range from about 10 to about 30 percent by volume. In another embodiment, the water-based gel comprises an absorbent polymer in the range from about 1 to about 10 percent by volume. In another embodiment, the bactericidal compound has a pH in the range from about 6.0 to about 6.5. In one embodiment, the cutaneous thermal wound comprises a bacterial infection. In one embodiment, the cutaneous thermal wound has a bacterial infection. In one embodiment, the cutaneous thermal wound comprises a noninfected burn wound. In another embodiment, the bacterial infection comprises a multi-drug resistant (MDR) gram-negative bacterial infection. In another embodiment, the bacterial infection is a MDR gram-negative bacterial infection. In another embodiment, the MDR gram-negative bacteria comprises Pseudomonas aeruginosa. In another embodiment, the MDR gram-negative bacterial infection is caused by Pseudomonas aeruginosa. In one embodiment, the bactericidal compound comprises from about 1 to about 5 mM of Na₂-EDTA and from about 20 to about 40 mM of NaNO₂. In another embodiment, the bactericidal compound comprises about 2 mM of di-sodium EDTA (Na₂-EDTA) and about 30 mM of NaNO₂. In another embodiment, the bactericidal compound comprises from about 20 to about 40 mM of Na₂-EDTA and from about 400 to about 600 mM NaNO₂. In another embodiment, the bactericidal compound comprises about 33 mM of Na₂-EDTA and about 500 mM NaNO₂. In one embodiment, the NaNO₂ is acidified NaNO₂. In another embodiment, the bactericidal compound has a ratio of acidified NaNO₂ (A-NO₂—) and Na₂-EDTA to water-based gel of about 1:100 (w/w). In one embodiment, the bactericidal compound comprises AB569. In another embodiment, the bactericidal compound is in a composition comprising AB569 and a water-based gel. In another embodiment, the water-based gel comprises Solosite®. In one embodiment, the subject is a mammal. In one embodiment, the subject is a human.

Also provided in present disclosure is a composition comprising a therapeutically effective amount of acidified NaNO₂ (A-NO₂⁻) and Na₂-EDTA, and a water-based gel. In one embodiment, the water-based gel comprises glycerol, an absorbent polymer and water. In another embodiment, the water-based gel comprises glycerol in the range from about 10 to about 30 percent by volume. In another embodiment, the water-based gel comprises an absorbent polymer in the range from about 1 to about 10 percent by volume. In another embodiment, the bactericidal compound has a pH in the range from about 6.0 to about 6.5. In one embodiment, the bactericidal compound comprises from about 1 to about 5 mM of Na₂-EDTA and from about 20 to about 40 mM of NaNO₂. In another embodiment, the bactericidal compound comprises about 2 mM of di-sodium EDTA (Na₂-EDTA) and about 30 mM of NaNO₂. In another embodiment, the bactericidal compound comprises from about 20 to about 40 mM of Na₂-EDTA and from about 400 to about 600 mM NaNO₂. In another embodiment, the bactericidal compound comprises about 33 mM of Na₂-EDTA and about 500 mM NaNO₂. In one embodiment, the NaNO₂ is acidified NaNO₂. In another embodiment, the bactericidal compound has a ratio of acidified NaNO₂ (A-NO₂—) and Na₂-EDTA to water-based gel of about 1:100 (w/w). In one embodiment, the composition comprises AB569. In another embodiment, the composition comprises AB569 and a water-based gel. In another embodiment, the water-based gel comprises Solosite®. In one embodiment, the subject is a mammal. In one embodiment, the subject is a human.

In one embodiment, the present disclosure uses AB569, delivered topically, to prevent the attachment and growth of bacteria by creating an environment that is not permissive for bacteria survival. In another embodiment, the present disclosure uses AB569 to modulate the wound bed for positive wound healing outcomes.

When AB569 is exposed to infected burn wounds according to the present disclosure, it triggers significant alterations in bacterial essential gene transcription that are critical for survival and growth. In contrast, genes related to inflammation, angiogenesis, epithelial cell proliferation, and ECM composition, will both increase and decrease due to AB569 exposure during burn wound healing.

### Delivery Vehicle

To deliver AB569 topically to burn wounds, the present disclosure comprises a delivery vehicle. In one embodiment, the delivery vehicle comprises a water-based gel. In one embodiment, the present disclosure uses a compound of AB569 and a water-based gel. In another embodiment, the water-based gel comprises Solosite®. In one embodiment, the compound of AB569 and water-based gel has a pH between 6 and 6.5. In one embodiment, the water-based gel comprises glycerol, an absorbent polymer and water. In another embodiment, the water-based gel comprises glycerol in the range from about 10 to about 30 percent by volume. In another embodiment, the water-based gel comprises an absorbent polymer in the range from about 1 to about 10 percent by volume. In one embodiment, the compound has a ratio of AB569 to water-based gel of about 1:100 (w/w). In one embodiment, the delivery vehicle is suitable for topical application.

### Experimental Results

The inventors conducted a series of experiments, described in detail below. These experiments revealed four key findings: (i) AB569 was effectively formulated as a gel and its components did not impede the release of bactericidal and wound healing levels of NO from A-NO₂—, and the Gram-negative membrane permeabilizing and perturbing properties of Na₂-EDTA, (ii) prophylactic application of AB569 gel likely prevented the colonization and establishment of PA infection in burn wounds, (iii) AB569 along with Solosite vehicle (SS) hastened the wound healing process more than SS treated wounds, and (iv) AB569 treatment modulates the wound bed by altering the expression of inflammatory cytokines.

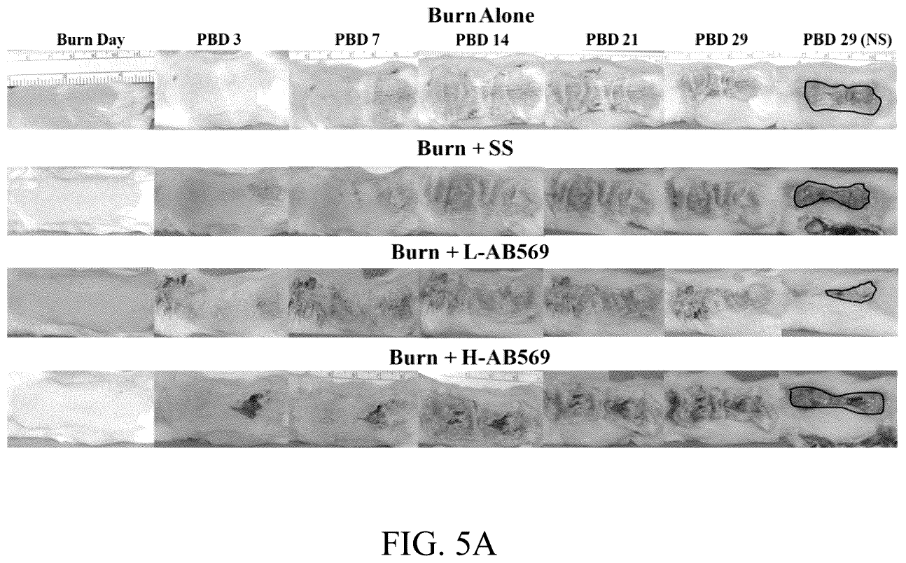

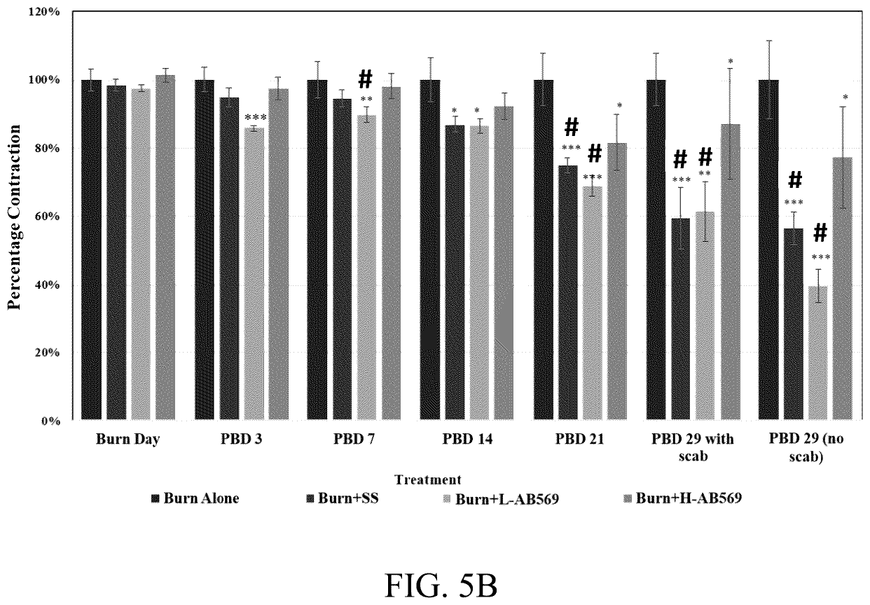

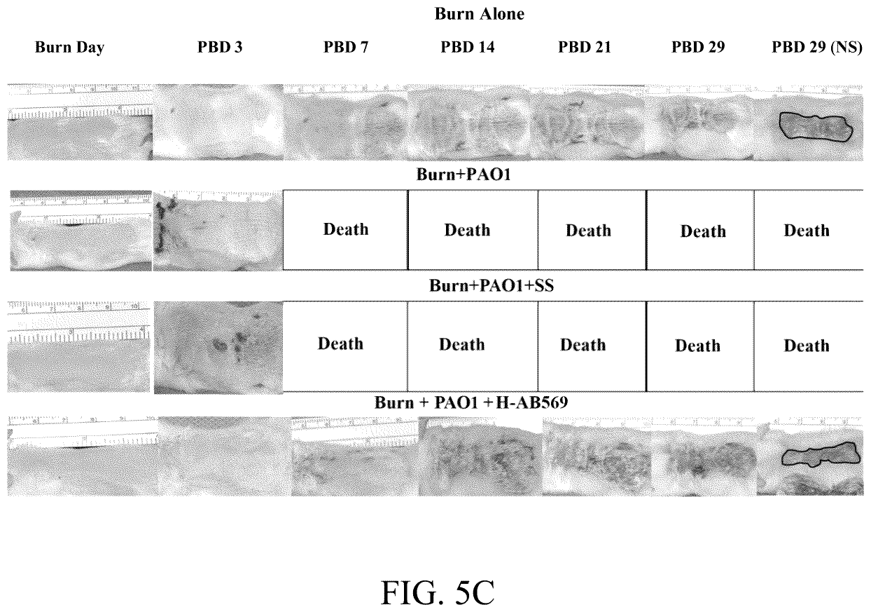

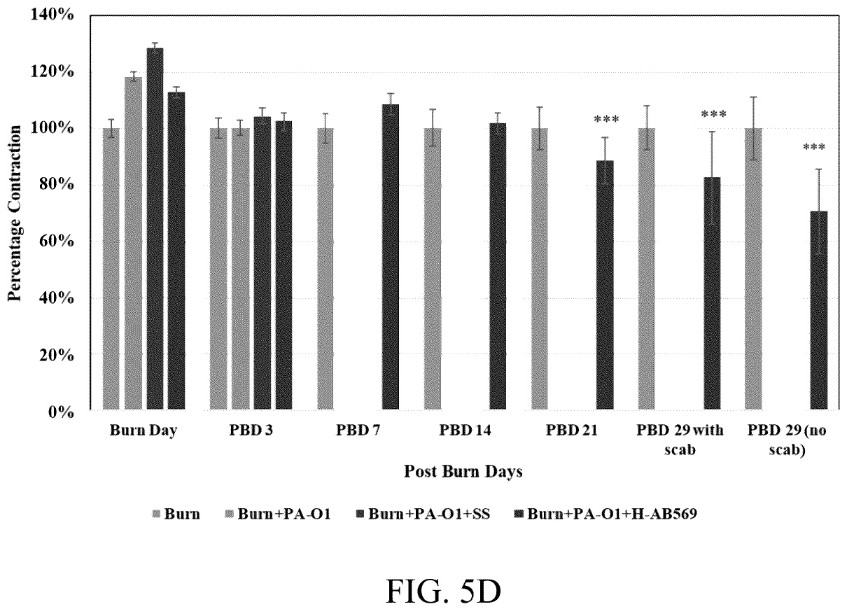

Inflammation plays an integral role in the healing of burn wounds as it influences the sequalae of events necessary for success of this vital process. Prolonged inflammation leads to poor scarring outcomes resulting in hypertrophic scar formation. However, the present results clearly point to two different strategies for treatment. After following burn wounds for a period of 30 days to determine the effect of AB569 on uninfected wounds, a dramatic and significant reduction in wound size was noted from post burn day 3 in uninfected wounds treated with L-AB569 (see FIG. 5A and FIG. 5B). Surprisingly, H-AB569 did not hinder wound closure in uninfected wounds but a significant difference from the untreated wounds was observed from post burn day 21 (FIG. 5A and FIG. 5B). In contrast, in infected animals, H-AB569 treated infected wounds not only eliminated the PA infection but enhanced wound closure (FIG. 5C and FIG. 5D), while such a significant difference in wound closure was not observed using L-AB569 treated infected wounds. Without being bound by theory, one reason may be that infected wounds required higher concentration of H-AB569 to clear infection and thereby also provides beneficial attributes for wound closure. Significantly, animals treated with AB569 formed a scab covering the underlying healing tissue similar to a cap. The scab formation potentially prevented the dehydration of the healing skin underneath, to protect from infections, and to prevent any entry of contaminants from the external environment. Scab formation can also be attributed to the efficient wound contraction seen in treated groups. Histopathological findings show that there was no significant difference in the influx of inflammatory cells between the treated groups in comparison to untreated burn wounds early (post burn day 4) during the wound healing process. Yet, analysis of postburn wound tissue harvested on post-burn day 30 showed less influx of inflammatory cells in AB569 treated group and improved collagen content and organization.

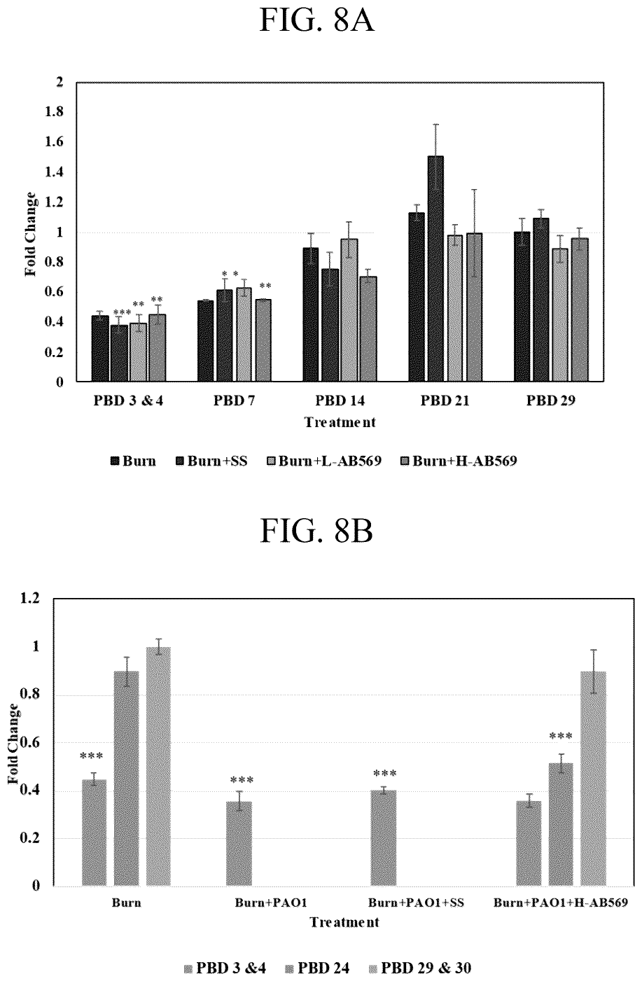

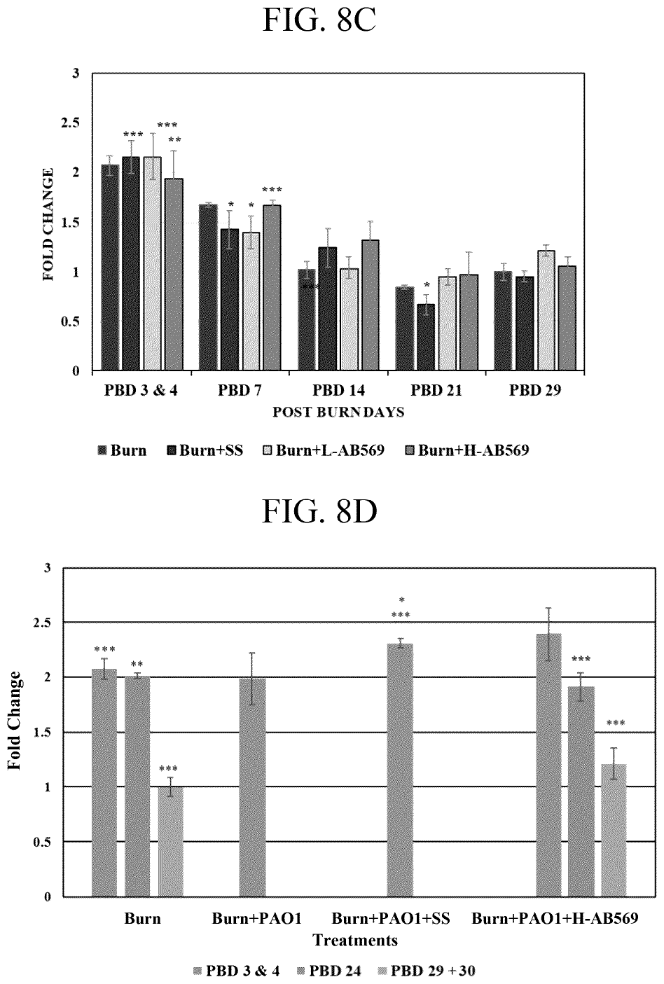

In addition, the inventors found that AB569 treated wounds decreased the expression of pro-inflammatory cytokines IL-6 and IL-10 and increased the expression of the anti-inflammatory cytokine IL-10 and immunomodulatory cytokine G-CSF on post-burn day 29 indicating that inflammatory response is not exacerbated in the healing wounds (FIG. 8B-E). G-CSF, a hematopoietic cytokine and potent stem cell mobilization agent, has been shown to play a central role as a regulator of the “genomic storm” driving divergent innate and adaptive immune responses after traumatic injury. The inventors noted an increased expression of G-CSF in both infected and uninfected wounds which may have positively driven faster clearing of the infection and wound healing. The decreased expression of IL-6 and IL-10 in non-treated wounds show that these two cytokines can work in concert in prolonging the inflammatory response and thereby triggering the wounds to be chronic and non-healing. Collectively, the inventors' results show that L-AB569 may be useful for prophylaxis of burn wound infection, while H-AB569 removes the infection while still enhancing wound closure.

The present disclosure shows a novel, non-toxic bactericidal drug formulation for the treatment of burn and other skin infections. AB569 represents a unique agent that has the potential to mitigate infection and accelerate the process of wound healing in burn and other infectious settings. The concentrations of AB569 were not only bactericidal against PA in vitro and during burn infection, but also dramatically enhanced the process of wound healing. The development of the present subject matter may have dramatic implications to global health, especially in burned patients. The present disclosure provides a positive impact on the development of non-antimicrobial approaches or as an adjuvant for wound treatment and management for civilian and military populations. It will also lessen the economic burden that MDR organisms are currently taxing the global health-care market.

Hereinafter are provided examples of specific embodiments and implementations for performing the methods and uses of the present disclosure. The examples are provided for illustrative purposes only, and are not intended to limit the scope of the present disclosure in any way:

### EXAMPLES

### Example 1

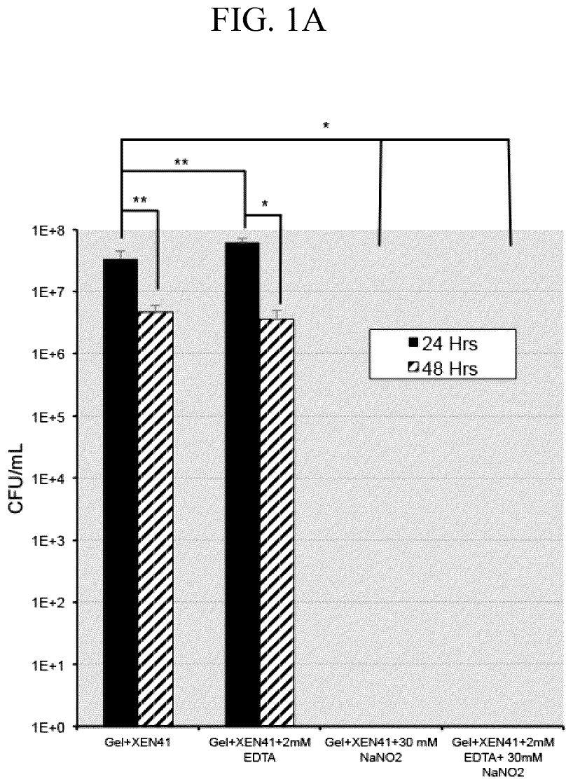

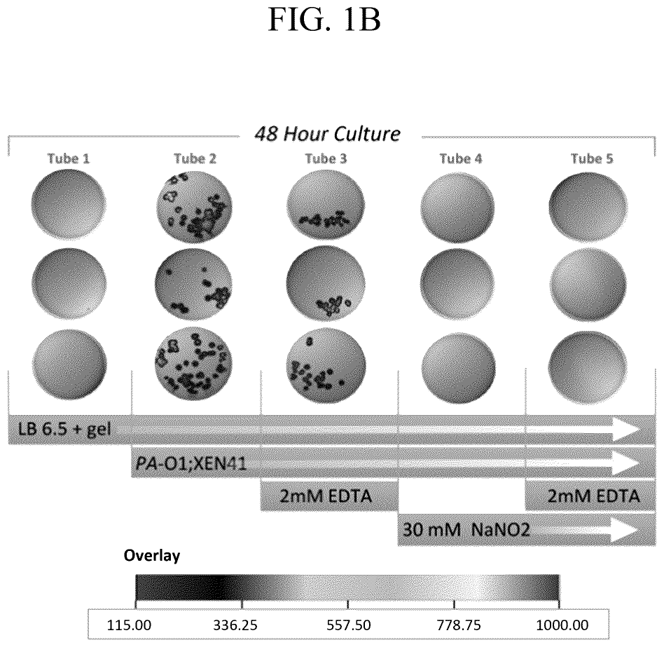

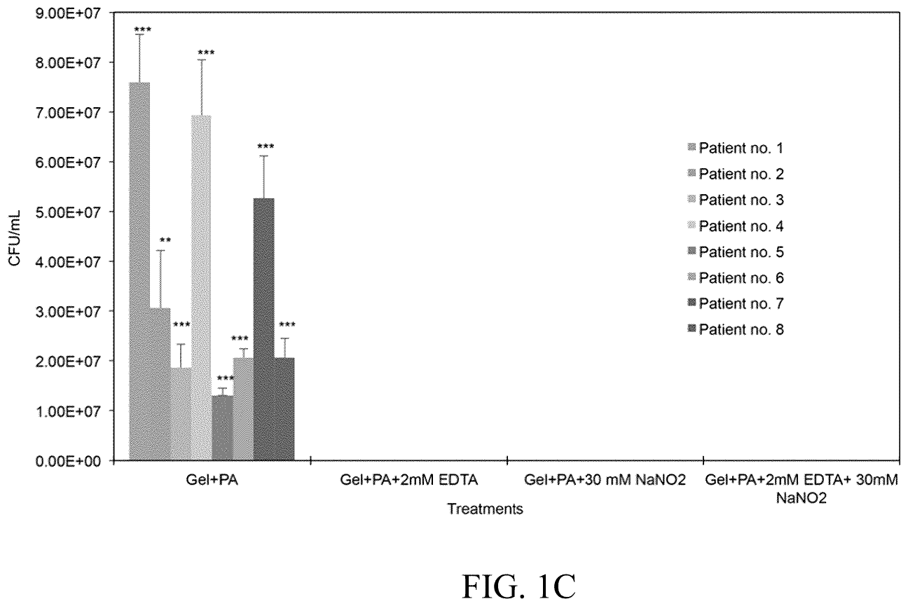

The efficacy of a formulation according to the present disclosure was tested in vitro using an overnight culture of bioluminescent PA Xen41, diluted in LB media (pH 6.5) along with a gel (Solosite®, herein SS) at a 1:100 dilution. Bioluminescent PA was used for both in vitro and in vivo experiments. Interestingly, gel in combination with 2 mM Na₂-EDTA exhibited significant killing of the pathogenic bacteria, although some luminescence was observed after 48 hours. In contrast, the gel in combination with either 30 mM NaNO₂ or 2 mM Na₂-EDTA plus 30 mM NaNO₂, completely killed PA and therefore bioluminescent signals (FIG. 1A and FIG. 1B).

### Example 2

The efficacy of the AB569 gel formulation was tested on different clinical strains of PA isolated from human burn patients with varying sensitivity and resistance to antibiotics using a broth based killing assay. The inventors found that AB569 at a concentration of 2 mM Na₂-EDTA and 30 mM NaNO₂ killed PA, irrespective of their antibiotic sensitivity and resistance (FIG. 1C). Interestingly, 2 mM Na₂-EDTA alone showed efficient killing of clinical strains of PA. Taken together, these data show that the topical-based gel does not interfere with the functioning of the active components of AB569 and it is an effective anti-microbial agent.

### Example 3

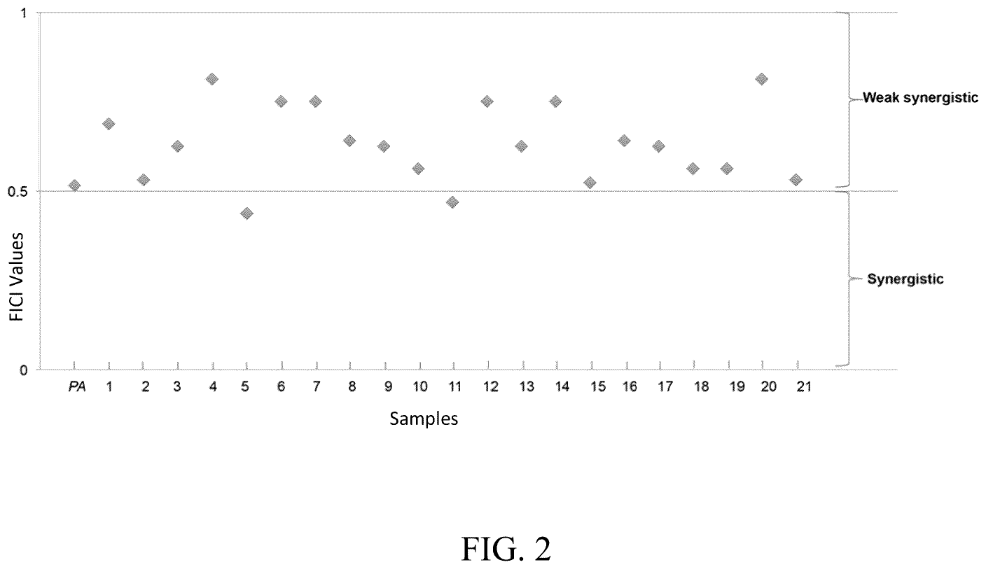

The inventors further confirmed the bactericidal activity of AB569 gel against various clinical strains of PA isolated from burn wound patients by synergy measurements using the checkerboard analysis. Interestingly, results showed a synergistic effect of AB569 on some of the burn wound PA strains (Fraction Inhibitory Concentration Index (FICI)<0.5) and on the other PA clinical strains and the PAO1 strain either displayed weak synergy (FICI<1) or exhibited an additive effect (FICI>1) (FIG. 2). Overall, AB569 killed the clinical strains along with the bioluminescent PA strain. The assay showed that all of the clinical strains of PA tested and PA-01 exhibited weak synergy and overall AB569 efficiently killed all the PA tested. FICI=Fractional inhibitory concentration index. FICI<0.05 is synergistic; 0.5<FICI<1 is weak synergistic.

### Example 4

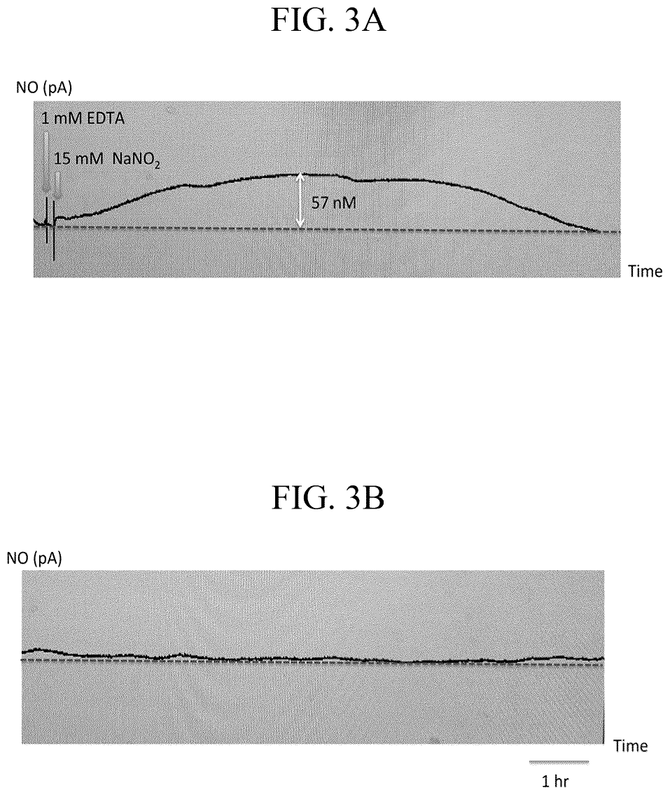

Critical pharmacokinetic studies were performed. To ascertain that the gel does not interfere with the release of nitric oxide (NO) from acidified NaNO₂ (A-NO₂—), NO polarographic measurements were performed using an ISO-NOP probe linked to an Apollo 4000 detector. The production of NO from the combination of bacterial culture medium (L-broth), 10% SS gel and AB569 (1 mM Na₂-EDTA, 15 mM A-NO₂—) was observed for ˜8 hours at a maximum concentration of bactericidal levels of NO (57 nM, FIG. 3A and FIG. 3B) indicating that the gel does not interfere with the production of NO by AB569.

### Example 5

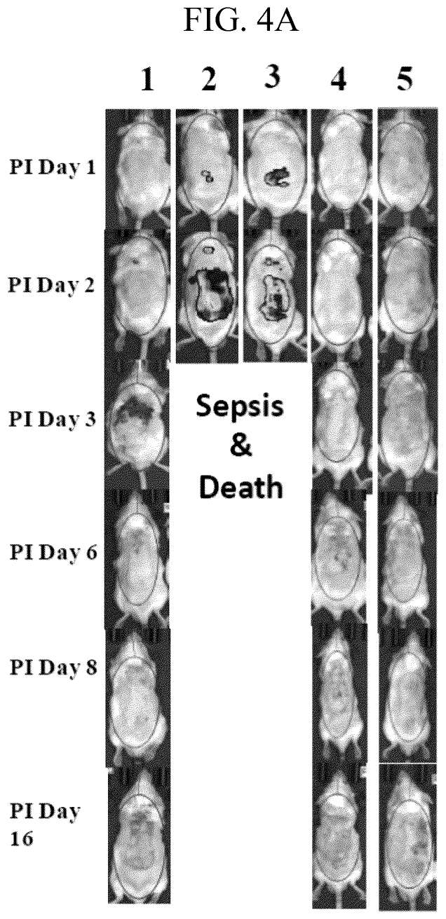

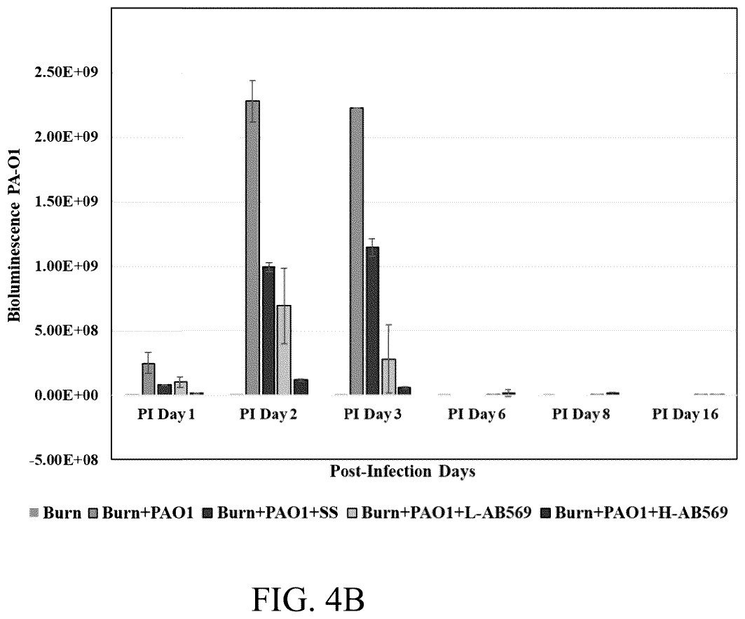

To determine the efficacy of AB569 in killing PA in an infected burn wound, a scald burn wound model is utilized. The infected scald burn wound model was established in CD-1 mice weighing between 30-40 g (6-8 weeks of age). Mice were anesthetized with 4.5% inhaled isoflurane in oxygen. Hair was then clipped from their dorsal surface. The mice were placed in a template that exposed 28% of their backs and immersed in 90° C. water bath for 9 s producing a well-demarcated full-thickness scald burn injury. Immediately following scald burn mice were resuscitated with 1.5 ml of sterile saline intraperitoneally and allowed to recover on a 42° C. heating pad. Sham mice underwent similar anesthetic and hair clipping, were immersed in room temperature water, and then resuscitated with 1.5 ml of sterile saline intraperitoneally. Twenty-four hours after creating the burn wounds, wounds were infected by topically inoculating with 200 μl of 2e4 CFU bioluminescent PA-Xen41. The establishment of PA colonization and infection on the skin burns was determined using the IVIS imaging system. These findings clearly indicate the presence of burn wound infection in skin between 24-48 h. The inventors also found that the topical application of AB569 gel-formulation in two different doses, 30 mM NaNO₂ and 2 mM Na₂-EDTA (low dose) and 500 mM NaNO₂ and 33 mM Na₂-EDTA (high dose), pH 6.0-6.5, eliminated infection with the high dose being more robust (FIG. 4A and FIG. 4B). FIG. 4B shows a quantification of photon intensities of bacterial burden showing significant reduction in photon intensities of PAO1 XEN41 in animals treated with low and high AB569. For FIG. 4A and FIG. 4B: 1−Burn alone (n=2); 2−Burn+PAO1 (n=7); 3−Burn+PAO1+SS (n=7); 4-Burn+PAO1+L-AB569 (n=12) and 5 Burn+PAO1+H-AB569 (n=23). PBD; post-burn days, L-AB569; Low AB569, H-AB569; High AB569; SS-Solosite. Treatments initiated immediately after burn injury, followed by the administration at 24 h before infection and continued until post-burn day 7, were found to inhibit the growth of PA and possibly attachment properties. After several iterations of compounding AB569 using the low and the high doses of AB569 specified above, the inventors determined that an appropriate consistency for topical gel application could be obtained. At higher or lower doses, the formulation lost its viscosity and was not suitable for topical application.

### Example 6

To assess the impact of AB569 on the localized response of burn wound tissue without PA infection, initial studies focused on grossly examining wound contraction in various treatment groups compared to untreated burn wounds (FIG. 5A). Gross images of the wounds were captured, and analysis of wound closure was performed using NIH Image J. Wounds were photographed weekly using a standard digital camera. These results indicate that by post burn day 29 without scab, SS alone (57±4.8%) and SS plus L-AB569 treated wounds (40±5%) triggered significant wound contraction. In FIG. 5B and FIG. 5D, rate of wound closure in uninfected treated burn wounds was measured by Image J from burn day to PBD 29. The data is normalized to burn for each post burn day. The student's t-test compared the asterisk(s) (*) p-values to burn day and the number sign (#) p-values to high AB569, on respective days. (Burn: n=20; H-AB569: n=23; SS: n=22; L-AB569: n=20; PAO1: n=10; PAO1+SS: n=4).

Interestingly, the wound contracting ability of the SS plus H-AB569 (77±4%) was less than SS alone. It should be noted, however, that the L-AB569 formulated with SS significantly enhanced wound contraction relative to SS alone by greater than 17% (p-value of 0.013, FIG. 5B). Thus, for the first time, the inventors have demonstrated that SS alone has wound healing benefits due likely to its hydrating of the extracellular matrix (allantoin) and antimicrobial/bacteriostatic (methylparaben, propylparaben, benzyl alcohol) properties. However, when SS was admixed with NaNO₂ and Na₂-EDTA, the effect was dramatically enhanced by more rapid wound closure and increased mouse survival. Another clinically relevant finding of these experiments was the enhancement of wound closure observed in infected wounds treated with H-AB569, while infected and infected+SS mice all died post-burn day 3 (FIG. 5C). Furthermore, a significant enhancement in wound healing was observed in PA wounds treated with H-AB569 (71±15%) on PBD 29 showing a clear reduction in wound size (FIG. 5D).

### Example 7

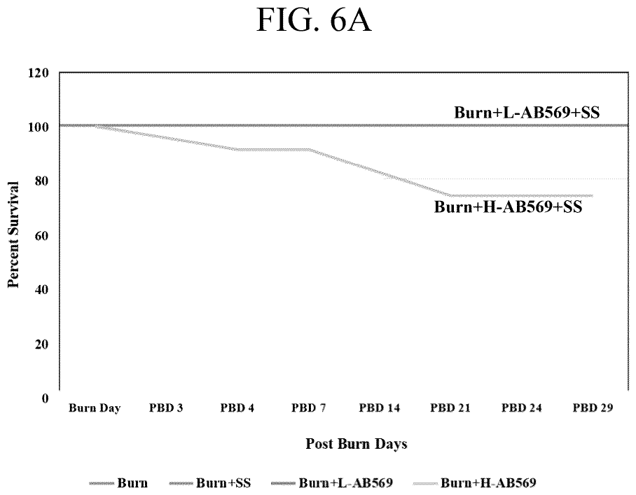

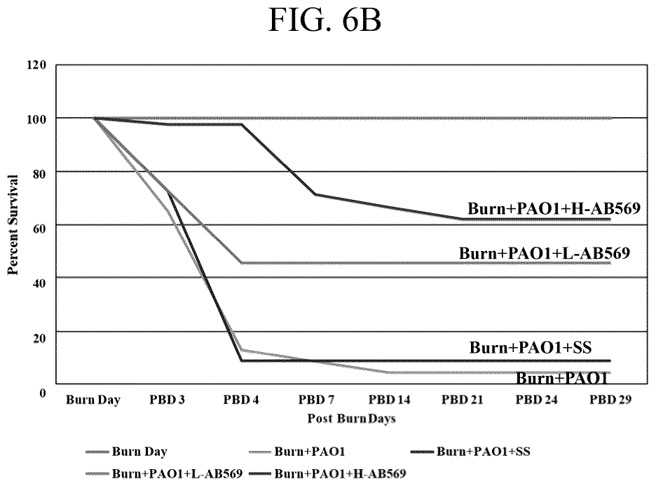

Full thickness scald burn injured animals that were uninfected, uninfected and treated with SS, and uninfected and treated with L-AB569 formulated with SS showed a 100% survival rate (FIG. 6A). Uninfected burn wounds with H-AB569 formulated with SS showed 91% survival on post-burn day 7, 82% survival rate on post-burn day 14, and 74% survival on post-burn day 29. H-AB569 also enhanced wound closure/contraction. In some cases, the inventors noted that there could be an increased risk of death upon multiple treatments. However, animals infected with PA and treated with L-AB569 formulated with SS showed 46% survival rate from post-burn days 4-29. In contrast, infected animals treated with H-AB569 in SS exhibited a 97% survival rate on post-burn day 4, and 62% survival on post-burn day 29. Untreated burn wound infected animals and infected burn wounds treated with SS alone succumbed to death except for one animal within 72-96 hours (FIG. 6A and FIG. 6B). Thus, the inventors' remaining focus was only on H-AB569 in PA infected wounds as the survival rate was higher when compared to infected wounds treated with L-AB569.

### Example 8

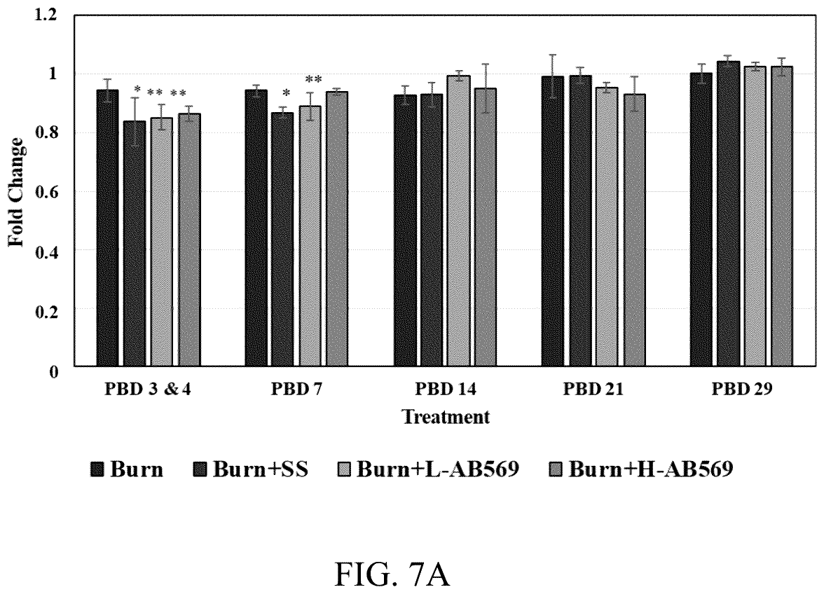

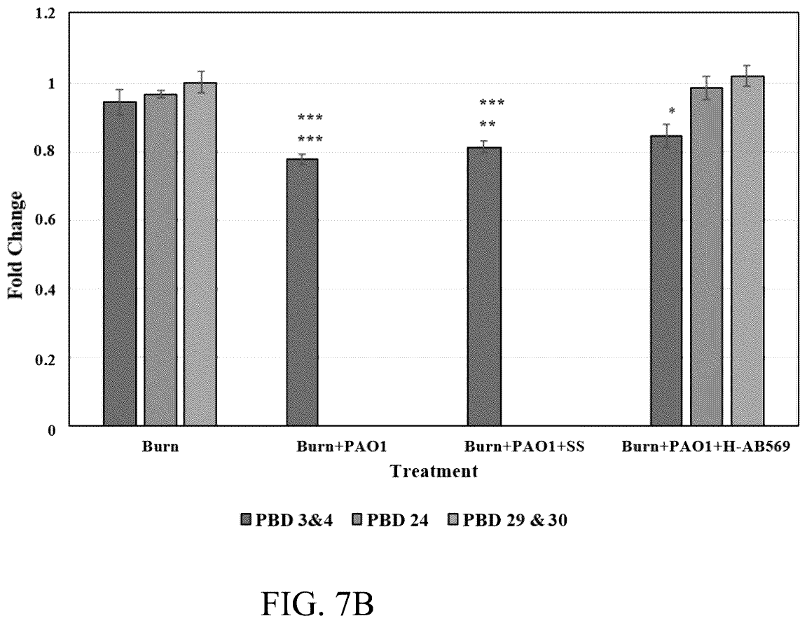

Since body weight is an index of an animal's overall health after burn injury, animal weights were recorded daily post-burn. A significant reduction in the body weights of the uninfected burn wound animals was observed on post-burn day 3, but the animals regained their weight by post-burn day 21 (FIG. 7A). In contrast, animals with infected burn wounds treated either with H-AB569 formulated in SS or treated with SS alone showed a significant weight loss on post-burn day 3, and the latter died within this period. In contrast, H-AB569-treated infected wounds showed a progressive increase in body weight from post-burn day 24 to post-burn day 29 (FIG. 7B).

### Example 9

AB569 protects infected burn wound animals by altering spleen function. To ensure that AB569 treatment of the murine burn wounds did not elicit any systemic side effects, the relevant sepsis-related organs harvested from sacrificed animals were carefully screened using several important health parameters. First, there were no significant changes in the size, shape and weight of the liver, lungs, and kidneys. However, significant changes were observed in both the size and weight of the spleen. In both untreated and treated uninfected burn wounds, the spleens of the animals were enlarged, and increased in weight progressively from post-burn day 3 to day 29 (FIG. 8A). Conversely, PA-infected animals treated with H-AB569 which survived infection showed significant increases in spleen weights and enlargement from post-burn day 3 to post-burn day 29 (FIG. 8B). These results show that an increase in spleen size may indirectly protect the animal from trauma and infection, especially given that two important functions of the spleen are microorganism clearance and removal of antigens. Similarly, when the body weight to spleen ratio was calculated in the infected and uninfected wounds treated or non-treated with AB569, the smaller the ratio indicated that the animals had an improved likelihood of survival (FIG. 8C and FIG. 8D). Thus, these results show that the smaller the spleen size is correlative with an increased chance of death.

### Example 10

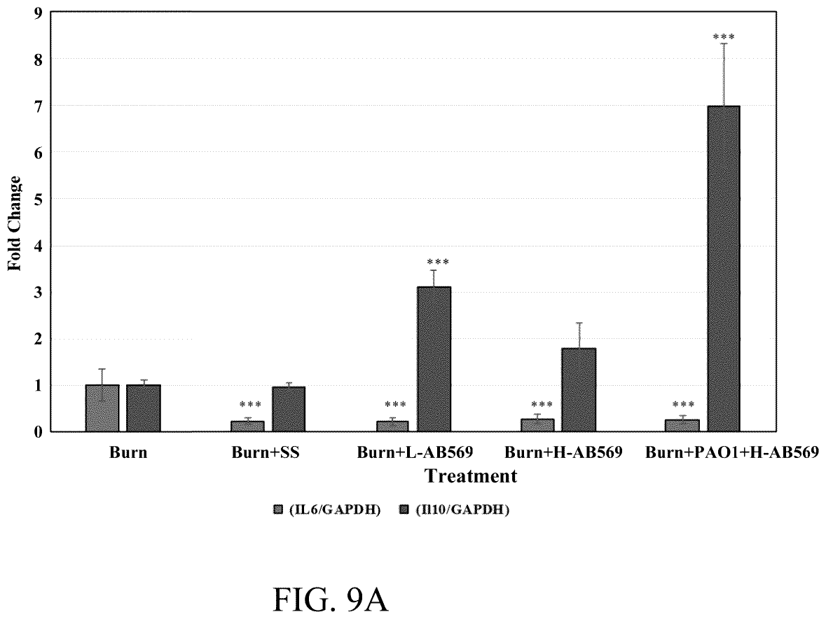

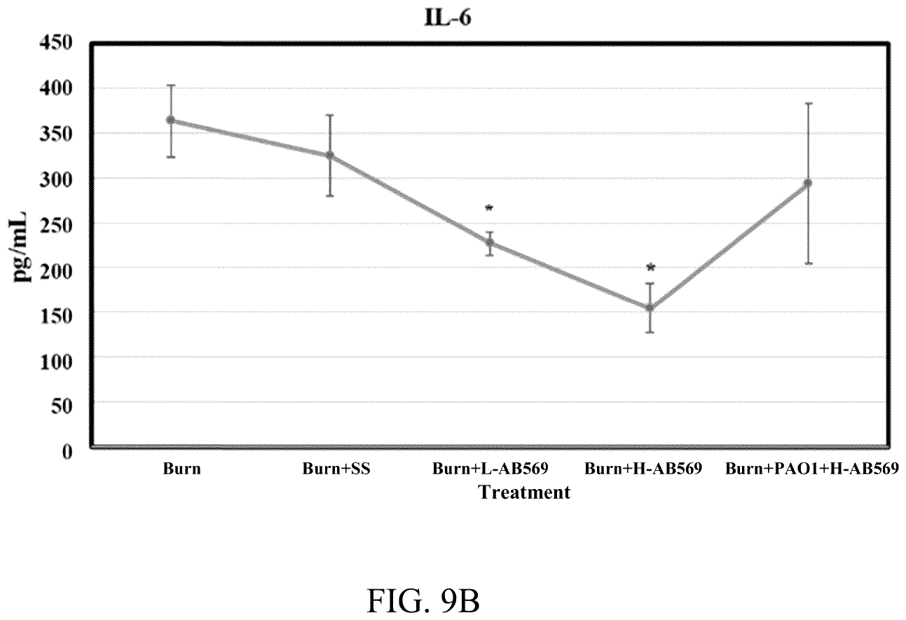

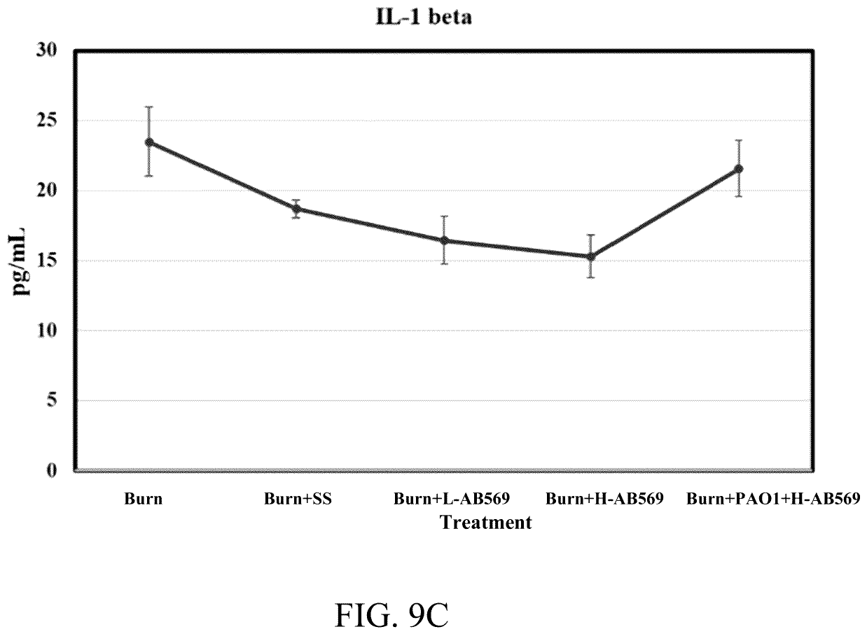

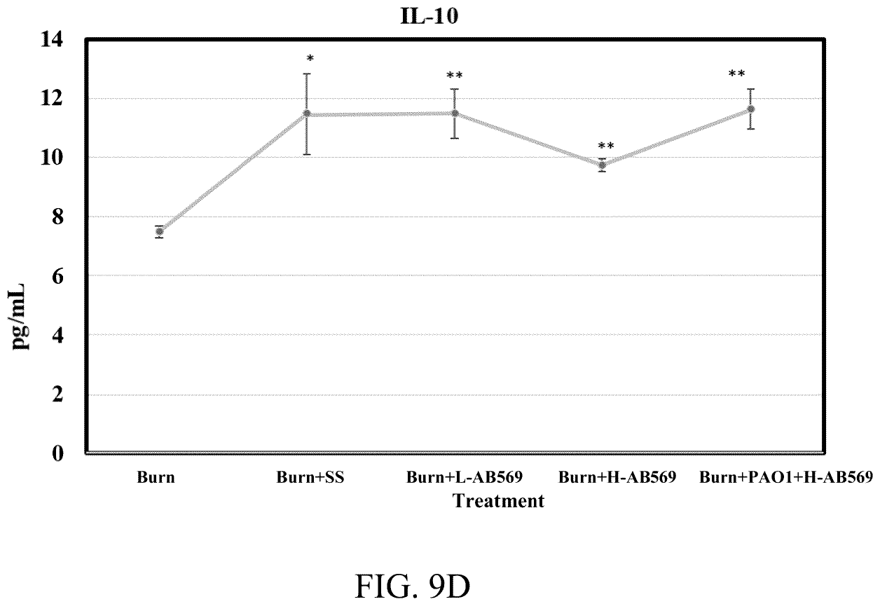

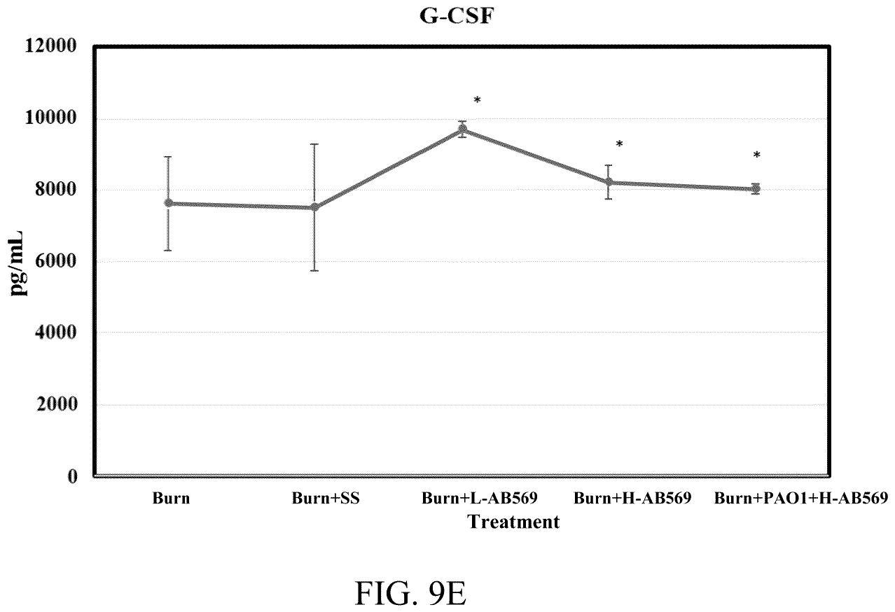

AB569 significantly alters the mRNA and protein expression levels of IL-6 and IL-10 in burn wounds. Burn injury elicits excessive inflammation that often lasts for several days. Hence, the inventors were next interested in determining whether the influx of inflammatory cytokines to the wound was altered due to topical administration of AB569 in the days post-injury. Interestingly, mRNA expression of IL-6 was significantly reduced in all of the treatment groups in comparison to the untreated burn wounds (FIG. 9A). Interestingly, SS-treated wounds also exhibited decreased expression of IL-6. In contrast, IL-10 expression was significantly elevated only in uninfected wounds treated with L-AB569 and PA infected wounds treated with H-AB569 (FIG. 9A). The inventors examined the levels of key serum cytokines by multiplex analysis of serum collected on post burn day 29. The inventors determined that the treatment with AB569 substantially reduced the levels of pro-inflammatory cytokine IL-6 in both L- and H-AB569 treated uninfected wounds (FIG. 9B). Further, the levels were decreased in the PA infected wounds treated with H-AB569, trending towards statistical significance relative to burn alone animals. With regards to IL-1, though a statistical significance was not observed in treated animals, levels were still low when compared to burn alone and SS treated animals (FIG. 9C). One of the key findings was a significant increase in the levels of IL-10 in all the treatment groups including SS treated animals (FIG. 9D). Interestingly, levels of G-CSF, an immunomodulatory cytokine, was significantly reduced in uninfected burn wounds treated with L- and H-AB569 (FIG. 9E).

### Example 11

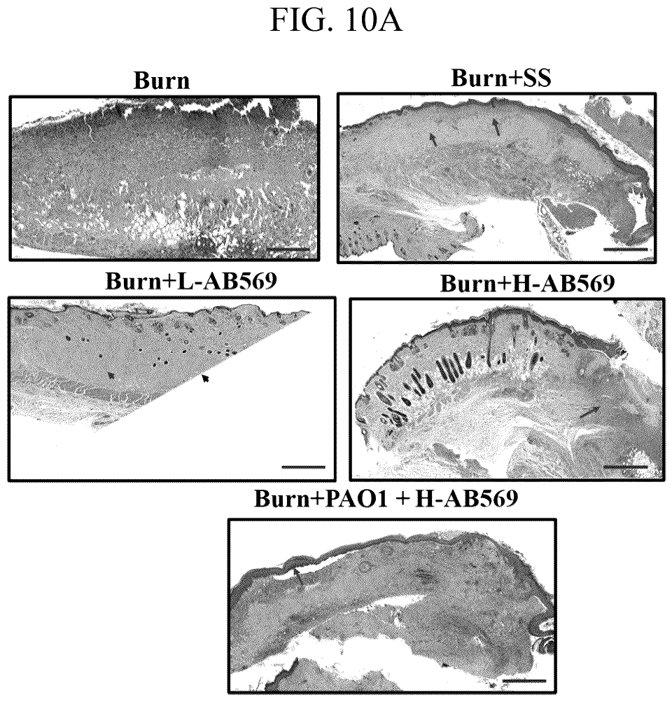

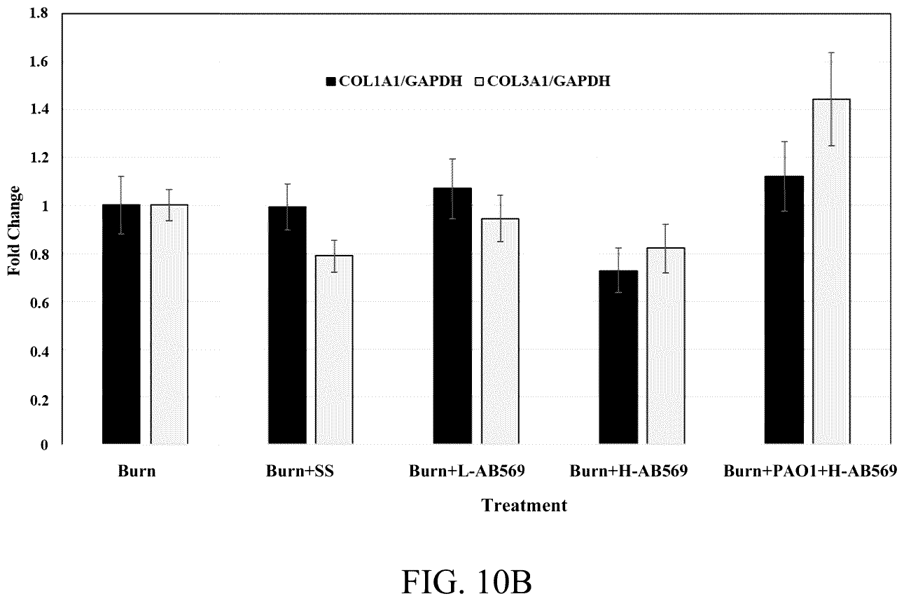

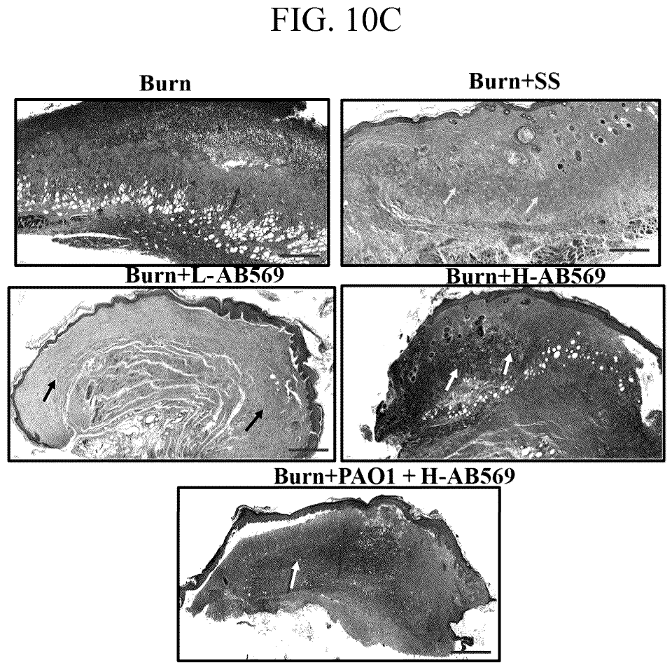

AB569 alters inflammatory status and collagen expression but did not alter the mRNA expression levels of type I and type III collagens. Gross images of H&E stained sections showed significant difference in burn wounds treated with L-AB569. There was an increased prevalence of mature fibroblasts and organized collagen deposition in the L-AB569 (arrows) treated group in comparison to the untreated group with less inflammatory cells (FIG. 10A). H&E stained wound sections from both control and treated groups were evaluated at 4 weeks postburn by pathology via a semiquantitative histological assessment using several parameters that included inflammation, fibrin exudate, presence of fibroblasts, collagen deposition and capillary proliferation (Table 1). Wounds in the AB569 cohort demonstrated better overall wound healing and all wounds completely re-epithelialized. Wounds in all groups revealed the presence of scab. Beneath the scab, however, there was complete re-epithelization in wounds treated with H- and L-AB569. Interestingly, SS-treated wounds also showed complete re-epithelialization indicating that wound occlusion helps in rapid epithelial coverage. The cumulative histopathologic score of skin wounds with different treatments showed similar trends between the control and treatment groups, with a slight lower score observed in wounds treated with L-AB569. At the mRNA level, the inventors did not observe any significant differences in both type I and type III collagens in both treated and untreated groups (FIG. 10B). The Masson's trichrome stain used to evaluate the collagen content (FIG. 10C) indicating a higher deposition in wounds treated with H-AB569 when compared to L-AB569 and SS treated wounds. Of note, thin collagen fibrils with more organized collagen structure was observed in L-AB569 treated wounds in comparison to PA plus H-AB569 and H-AB569 treated groups.

TABLE 1
Histopathological Score of Burn Wounds in Different Treatments

| Histological differences | Burn | | Low | High | PA + High |
|---|---|---|---|---|---|
| seen on H & E stained sections | alone | SS | AB569 | AB569 | AB569 |
| Acute polymorpho nuclear leukocytes inflammation | 4 | 4 | 2 | 3 | 3 |
| (score 0-4 per wound) 0 = no inflammation; | | | | | |
| 1 = minimal inflammation; 2 = mild; | | | | | |
| 3 = moderate; 4 = marked inflammation | | | | | |
| Fibroblasts and capillary proliferation | 1 | 2 | 3 | 2 | 3 |
| (Score 0-3 per wound) 0 = no fibroblasts | | | | | |
| and blood vessels/capillaries; 1 = few | | | | | |
| fibroblasts with few capillaries; 2 = | | | | | |
| Intermediate presence of fibroblasts and | | | | | |
| presence of capillaries; 3 = Mature | | | | | |
| fibroblasts and several blood vessels | | | | | |
| Amount of collagen deposition between | 1 | 1 | 4 | 2 | 1 |
| fibroblasts (score 0-4 per wound) (0 = | | | | | |
| normal large amount; 1 = marked; 2 = | | | | | |
| moderate; 3 = mild; 4 = minimal amount) | | | | | |
| Total scoring of the wounds (Higher the | 6 | 7 | 9 | 7 | 7 |
| score signifies better healing outcome) | | | | | |

### Example 12

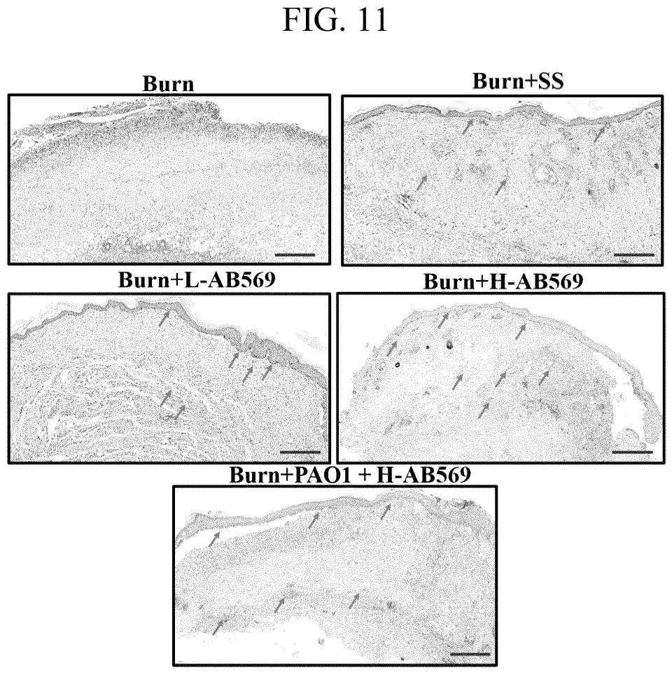

AB569 treatment promotes better epidermal restoration. The morphological findings in H&E stained sections show that in addition to complete wound closure, AB569 treated wounds also showed better epidermal restoration. The inventors stained the wounds sections with Ki67 to determine the rate of epithelial proliferation. Representative images of wounds from n=4 wounds in all the different groups are stained with Ki67 (pointed by the arrows) are shown in FIG. 11. Scale bars are noted in the images (black bars, lower right). Complete epithelial coverage was noted in all the treatment groups in comparison to burn alone and PAO1 infected wounds. PBD; post burn day, SS; Solosite, L-AB569; Low A1B569, H-A1B569; High A1B569. These data show a complete re-epithelialization in infected and uninfected wounds treated either with L- or H-AB569 (FIG. 11). The positive staining of Ki67 observed in the epidermis was significantly higher in wounds treated with L-AB569, whereas in the H-AB569 treated group, the proliferating cells were higher in both epidermis and dermis (FIG. 11). In PA infected animals non-specific staining of Ki67 was observed throughout the tissues and PA infected and H-AB569 treated animals showed significant staining in epidermis and some staining was also observed in dermis.

### Example 13

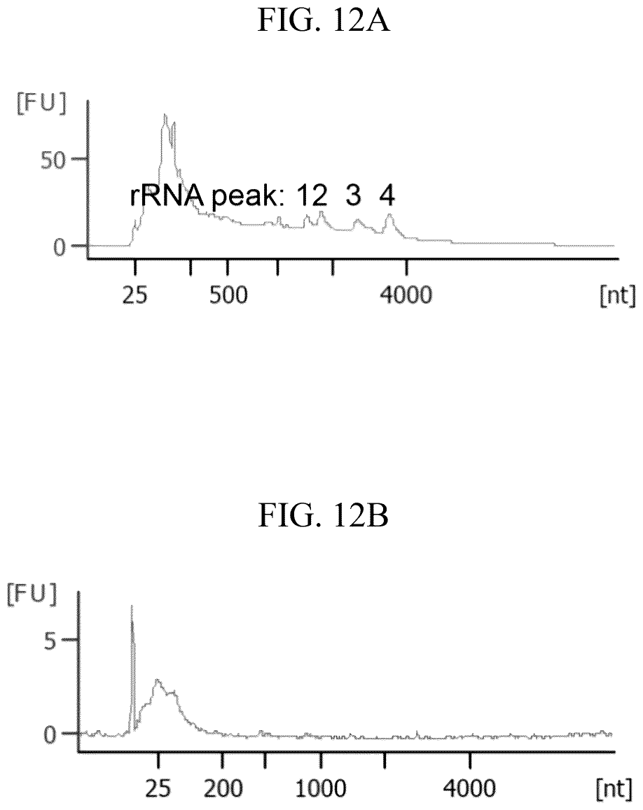

To assess the impact of the combinatorial application of NaNO₂ and Na₂-EDTA (AB569) on the localized response of the wound tissue, the inventors analyzed important wound healing parameters as well as extracellular matrix (ECM) protein changes in burn wound-infected mice treated with gel +/− AB569. These initial studies on grossly examining infected and non-infected burn wounds treated with a high dose of AB569 (500 mM NaNO₂ and 33 mM Na₂-EDTA) show rapid wound closure compared to untreated wounds (FIG. 12A and FIG. 12B) by post-burn day 14.

### Example 14

### Methods

### Strains, Media and Growth Conditions

All clinical isolates used in this study were obtained from the microbiology department of Shriners Hospitals for Children-Cincinnati. Bioluminescent PA-Xen41, derived from parental strain PAO1, was purchased from PerkinElmer (Waltham, MA). Luria broth (LB) media was composed of 10 g tryptone, 5 g yeast extract, and 5 g NaCl (and an additional 15 g Bacto-agar for LB agar) per liter (all chemicals were from Fisher Scientific). Tryptic Soy broth (TSB, Becton Dickinson) and TSB plus 1.5% agar (TSA) were prepared according to the manufacturer's instructions. All strains listed in Table 2 were grown with appropriate media plates or broth.

TABLE 2

| | MIC EDTA | MIC NaNO₂ | FIC EDTA | FIC NaNO₂ | | |
|---|---|---|---|---|---|---|
| Strains | (mM) | (mM) | (mM) | (mM) | FIC | Index (FICI) |
| PAO1 | 0.833 ± 0.289 | 18.667 ± 12.220 | 0.333 ± 0.144 | 4.083 ± 3.876 | 0.594 ± 0.136 | Weak Synergy |
| Burn #1-1 | 0.5 ± 0 | 26.667 ± 9.238 | 0.25 ± 0 | 5.333 ± 2.309 | 0.708 ± 0.072 | Weak Synergy |
| Burn #1 | 0.833 ± 0.289 | 18.667 ± 12.220 | 0.417 ± 0.144 | 4.167 ± 3.752 | 0.604 ± 0.130 | Weak Synergy |
| Burn #2-1 | 0.833 ± 0.289 | 26.667 ± 9.238 | 0.333 ± 0.144 | 6.667 ± 2.309 | 0.667 ± 0.144 | Weak Synergy |
| Burn #2 | 0.5 ± 0 | 14.667 ± 15.144 | 0.188 ± 0.108 | 7.333 ± 7.572 | 0.875 ± 0.217 | Weak Synergy |
| Burn #3-1 | 0.667 ± 0.289 | 21.333 ± 9.238 | 0.208 ± 0.072 | 5.333 ± 2.309 | 0.583 ± 0.144 | Weak Synergy |
| Burn #3 | 0.833 ± 0.289 | 24 ± 13.856 | 0.333 ± 0.144 | 6.667 ± 2.309 | 0.667 ± 0.289 | Weak Synergy |
| Burn #4-1 | 0.5 ± 0 | 21.333 ± 9.238 | 0.25 ± 0 | 9.333 ± 6.110 | 0.833 ± 0.144 | Weak Synergy |
| Burn #4 | 0.667 ± 0.289 | 13.333 ± 4.618 | 0.125 ± 0.108 | 2.75 ± 2.165 | 0.677 ± 0.126 | Weak Synergy |
| Burn #5-1 | 0.5 ± 0 | 21.333 ± 9.238 | 0.167 ± 0.072 | 2.667 ± 1.155 | 0.625 ± 0 | Weak Synergy |
| Burn #5 | 0.5 ± 0 | 13.333 ± 4.618 | 0.25 ± 0 | 4.667 ± 3.055 | 0.584 ± 0.191 | Weak Synergy |
| Burn #6-1 | 0.5 ± 0 | 13.333 ± 4.618 | 0.167 ± 0.072 | 2.333 ± 1.523 | 0.521 ± 0.036 | Weak Synergy |
| Burn #6 | 0.667 ± 0.289 | 18.667 ± 12.220 | 0.25 ± 0 | 5.333 ± 2.309 | 0.75 ± 0 | Weak Synergy |
| Burn #7-1 | 0.5 ± 0 | 21.333 ± 9.238 | 0.19 ± 0.09 | 6 ± 3.464 | 0.667 ± 0.144 | Weak Synergy |
| Burn #7 | 0.833 ± 0.289 | 24 ± 13.856 | 0.25 ± 0 | 6.667 ± 2.309 | 0.667 ± 0.289 | Weak Synergy |
| Burn #8-1 | 0.5 ± 0 | 21.333 ± 9.238 | 0.25 ± 0 | 0.75 ± 0.433 | 0.537 ± 0.023 | Weak Synergy |
| Burn #8 | 0.667 ± 0.289 | 16 ± 0 | 0.333 ± 0.144 | 2.167 ± 1.756 | 0.635 ± 0.109 | Weak Synergy |
| Burn #9-1 | 0.5 ± 0 | 21.333 ± 9.238 | 0.167 ± 0.072 | 4.666 ± 3.055 | 0.541 ± 0.191 | Weak Synergy |
| Burn #9 | 0.5 ± 0 | 13.333 ± 4.618 | 0.167 ± 0.072 | 5.333 ± 2.309 | 0.667 ± 0.144 | Weak Synergy |
| Burn #10-1 | 0.416 ± 0.144 | 16 ± 0 | 0.125 ± 0 | 4 ± 0 | 0.583 ± 0.144 | Weak Synergy |
| Burn #11-1 | 0.667 ± 0.289 | 32 ± 27.713 | 0.167 ± 0.072 | 8 ± 0 | 0.667 ± 0.315 | Weak Synergy |
| Burn #12-1 | 0.5 ± 0 | 26.667 ± 9.238 | 0.167 ± 0.072 | 6 ± 3.464 | 0.604 ± 0.130 | Weak Synergy |

### Checkerboard Assays for MIC and FIC Determination

96-well polystyrene plates were filled with 100 μl of LB, pH 6.5 and 10% SS hydrogel (Smith and Nephew, London, UK). Row A was filled with 100 μl of a 4× stock of Na₂-EDTA (16 mM) and Column 10 was filled with 4× NaNO₂ stock (256 mM), bringing both to a 2× concentration. Two-fold serial dilutions were performed such that a concentration gradient of each was created. Column 12 was filled with media to represent a negative control. An overnight culture of bacteria was adjusted to an OD600 of 0.5 in LB media. This was then diluted 1:1000 into fresh media, which was then added to the checkerboard plate (columns 1-11). This dilution was selected as it harbored ˜5×105 CFU/ml. Column 11 was the positive control. Plates were incubated at 37° C. The cell turbidity was determined using a 96 well plate reader after 24 hours inoculation. The threshold used for a positive cutoff to calculate MICs and FICs was 0.001 after blank (media) subtraction.

### Broth Based Killing Assay

Bioluminescent PA-Xen41 was grown overnight in LB media at 37° C. with shaking. Overnight cultures were diluted 100-fold into fresh LBN (LB-1% KNO3) 6.5 media and 5 ml aliquots were transferred to culture tubes. Final concentrations of 30 mM NaNO₂ and/or 2 mM Na₂-EDTA were added to each tube. Cells were grown anaerobically at 37° C. mimicking a mature biofilm. Samples were taken daily for 48 hours while the cells were still in the anaerobic chamber. Samples were serially diluted in PBS, pH 7.4, and 10 μl aliquot from each dilution placed on LB agar plates and grown aerobically overnight at 37° C. CFU were enumerated the next morning and converted to CFU/ml after multiplying by the dilution factor.

### Full-Thickness Scald Burn Wounds

Male and female CD-1 mice aged 8-10 weeks, 27-40 g, were housed singly after creation of burn wounds were obtained from Charles River Laboratories. Inc. (Wilmington, MA). This study was approved by the University of Cincinnati Institutional Animal Care and Use Committee (protocol #17-06-02-01). On the day of burn injury, animals were administered with buprenorphine SR 1 mg/kg one hour before the creation of burns. Animals were anesthetized, using 4% inhaled isoflurane in oxygen and a full-thickness, well-demarcated scald burn was created by placing a shaved mouse in a template exposing 28% of their dorsal surface, followed by immersion in a 92.3° C. water bath for 9 seconds. The mouse was then carefully removed with great care taken not to scratch their backs. The template used was a 60 ml syringe Kendall (cat. no. #1186000777 Tyco/Healthcare), Luer Lock Syringe with Tip Cap, Latex-Free made of polypropylene. Immediately after burn injury animals were resuscitated by subcutaneous administration of 1.5 ml of 0.9% saline and were placed on a 42° C. heating pad to recover.

Pseudomonas aeruginosa (PA) Infection

Bioluminescent PA-Xen41 (derived from parental strain PAO1; PerkinElmer®, Waltham, MA) was grown overnight in 5 ml of tryptic soy broth at 37° C. with shaking for 16-18 hr. On post-burn day 1, mice were anesthetized with 4% isoflurane, and 200 μl of 2×104 bioluminescent PA was topically inoculated on the wound using an inoculating loop.

### Preparation of AB569 Gel Formulation

Solosite® (herein, SS, Smith & Nephew, London, UK) a water-based gel was used as a delivery vehicle for AB569. The density of SS is approximately that of water, 1 g/ml, and, therefore, the volume of gel can be determined by mass (i.e., 1 gram of SS (Medline Industries, REF 449600; Northfield, IL) is 1 ml approximately). Two different concentration of AB569 was used for the in vivo experiments: (a) 2 mM Na₂-EDTA and 30 mM NaNO₂ (L-AB569) and (b) 33 mM EDTA and 500 mM NaNO₂ (H-AB569). The desired concentration of Na₂-EDTA and NaNO₂ was added to 1 ml SS and the pH adjusted to 6.0-6.5 using 1N HCl and pH was confirmed using pH test strips. The ingredients of AB569 were mixed with SS at the time they were applied to the wound. The entire 1 ml of the gel formulation was applied to each wound. PA infected and uninfected burn wounds were either treated or non-treated with an L- or H-AB569 gel formulation and with SS alone.

### Bioluminescent Imaging

All living animals underwent IVIS imaging system (Caliper Life Sciences, Waltham, MA) of their wounds on post-burn days 2, 3, 4, and 6 to monitor the growth of bioluminescent PA. All animals are anesthetized with inhaled 2% isoflurane for imaging (exposure time of 2 min). The IVIS camera was maintained at standard settings, as follows: imaging mode luminescent, exposure time Auto, binning medium, F/stop 1, Field of View D. Once compiled, images compared quantitatively for relative increase/decrease in bioluminescence using Xenogen Living Image® software.

### Tissue Harvesting

Animals were sacrificed approximately around post-burn day 29. If the animals exhibited signs of morbidity and pain, they were sacrificed usually occurring between 48-72 h after infection. Collected wound tissues were stored in 10% formalin for histological analyses and tissues stored in RNA later was stored in −80° C. for RNA extraction and qRT-PCR analyses.

### Histological Staining

Histological section analysis was performed on wound tissue to determine the inflammation, epidermal regeneration, and amount of collagen deposition. Thin sections (4 μm) were cut and stained with Hematoxylin and Eosin (H&E), Mason's trichrome stain and Ki67 staining. Stained slides were scanned into digital images with Thermo Fisher 3DHistech Panoramic DESK Scanner with 40× objective and images were viewed using CaseViewer and photographed. Sections were stained with Hematoxylin and Eosin (H&E) to assist in the analysis of infiltration of each cell type, and a subjective score (no, mild, moderate, or severe) was given taking into consideration of all live cells within the histological section. The presence of neutrophils, macrophages, and plasma cells were compared between treated and untreated groups. Masson's trichrome staining was performed to determine the differences qualitatively in the collagen content between the treated and untreated burn wound tissues. All sections were stained with Masson's trichrome at the same time to eliminate variations in staining.

### Ki67 Staining

Formalin-fixed, paraffin-embedded tissues were cut from representative blocks at a thickness of 4 μm, placed on a charged glass slide, and dried in an oven at 55° C. for 3 hr. Using the fully automated Leica NOND RXm, the tissue sections were deparaffinized and subjected to heat-induced epitope retrieval using the BOND Epitope Retrieval Solution (pH 6.0) for 20 min. Endogenous peroxidase activity was blocked using the Refine Detection Kit Peroxide Block solution. Immunohistochemical staining was performed using the Ki-67 Recombinant Rabbit Monoclonal Antibody (Thermo Fisher Scientific, (SP6) MA5-14520, 1:100). The staining was visualized using the Leica BOND Polymer Refine Detection Kit.

### RNA Isolation and Quantitative Real-Time RT-PCR (RT-qPCR)

Approximately 30-40 mg of tissue was weighed and homogenized with a bullet blender for 3 min. If not completely homogenized, it was blended for another 3 min and then spun down for 1 minute at 13,300×g. After the tissue was homogenized, total RNA was isolated from the burned wound tissue using the RNeasy MINI kit (Qiagen Inc, Valcenia, CA) following the manufacturer's instructions. The quantity and quality of RNA were determined by measuring the OD 260/280 ratio using an ND-100 spectrophotometer (Nanodrop Technologies Inc., Wilmington, DE) and by capillary electrophoresis using an Agilent 2100 BioAnalyzer (Santa Clara, CA). The RNA with a RIN value >7 was used for RT-qPCR assays.

Total RNA isolated (RNeasy Mini Kit, Qiagen Inc.) from burn wound tissue treated and untreated with AB569 and SS treated alone were subjected to RT-qPCR to determine the gene expression levels of collagen types 1 and 3 and MMP-9 genes. QIAshredder columns were used to homogenize samples followed by running the samples through the gDNA eliminator column to remove any genomic DNA present in the sample. cDNA was prepared using the superscript Vilo cDNA kit (Invitrogen/Thermo Fisher Scientific). Taqman Universal PCR Master Mix primers used for the following mouse gene products were purchased from Thermo Fisher.: GAPDH (Mm05724508_g1), Col3α1 (Mm01254476_m1), Col1α1 (Mm00801666_g1), and MMP9 (Mm00442991_m1), IL-6 (Mm00446190_m1) and IL-10 (Mm1288386_m1). Real-time PCR was performed using a StepOnePlus Real-Time PCR System using the following protocol: denaturation 95° C. for 10 min, then 40 cycles of amplification at 95° C. for 15 s followed by annealing and extension at 60° C. for 1 min. The comparative 2-ΔΔCT method was used to determine the expression levels of target genes after normalization to GAPDH expression. The data are presented as fold changes in relative expression levels compared to burn wound for both treated and non-treated infected and uninfected burn wounds ±SEM.

### Cytokine Determinations in Serum

For isolation of serum, whole blood was collected by cardiac puncture, allowed to clot on ice and centrifuged. Serum were frozen at −80° C. and cytokine concentrations were determined by using Milliplex™ Multiplex kits (MilliporeSigma, Darmstadt, Germany) according to manufacturer's protocol. Briefly, in a 96 well black plate, 25 μL sample in duplicate was incubated with 25 μl antibody coated beads overnight at 4° C. on a plate shaker. Plates were then washed 2 times using the BioTek 405 TS (BioTek, Winooski, VT) and 25 μL of secondary antibody was added and incubated at room temperature for 1 hour on while shaking. Finally, 25 μl of streptavidin-RPE was added directly to the secondary antibody and incubated for 30 min at room temperature with shaking. Plates were then washed 2 more times and 150 μl of sheath fluid was added. Plates were shaken for 5 min and then read using Luminex technology on the Milliplex Analyzer (MilliporeSigma, Darmstadt, Germany). Concentrations were calculated from standard curves using recombinant proteins and expressed in pg/ml. Data analysis were performed by the Research Flow Cytometry Core at Cincinnati Children's Medical Center.

### Wound Area Measurements

Images of the wounds were captured from the day the burn wounds were created. NIH Image J software was utilized to determine the extent of wound contraction in AB569 treated, SS treated and untreated groups.

### Statistical Analysis

Statistical analysis was performed using Student's t-test. P-value<0.05 was considered statistically significant. Student's paired t-test was used to compare the differences between the control and experimental groups.

The present disclosure is not to be limited in scope by the specific embodiments described herein, since such embodiments are intended as but single illustrations of one aspect of the disclosure and any functionally equivalent embodiments are within the scope of this disclosure. Indeed, various modifications of the disclosure in addition to those shown and described herein will become apparent to those skilled in the art from the foregoing description and accompanying drawings. It will be appreciated how various modifications may be made without departing from the disclosure. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. The citation of any reference herein is not an admission that such reference is available as prior art to the instant disclosure.

What is claimed is:

1. A method for treating a subject having a cutaneous thermal injury, said method comprising administering a therapeutically effective amount of a bactericidal compound topically to said injury, wherein the bactericidal compound comprises a therapeutically effective amount of acidified NaNO₂ (A-NO₂—) and Na₂-EDTA.

2. The method of claim 1, wherein a preventative effective amount of the bactericidal compound is administered.

3. The method of claim 1, wherein the bactericidal compound is in a composition that further comprises a water-based gel.

4. The method of claim 1, wherein the water-based gel comprises glycerol, an absorbent polymer, and water.

5. The method of claim 1, wherein the water-based gel comprises glycerol in the range from about 10 to about 30 percent.

6. The method of claim 1, wherein the bactericidal compound has a pH in the range from about 6.0 to about 6.5.

7. The method of claim 1, wherein the cutaneous thermal injury comprises a bacterial infection.

8. The method of claim 7, wherein the bacterial infection is caused by a multi-drug resistant (MDR) gram-negative bacteria.

9. The method of claim 8, wherein the MDR gram-negative bacteria comprises Pseudomonas aeruginosa.

10. The method of claim 1, wherein the bactericidal compound comprises from about 1 to about 5 mM of EDTA and from about 20 to about 40 mM of NaNO₂.

11. The method of claim 1, wherein the bactericidal compound comprises about 2 mM of EDTA and about 30 mM of NaNO₂.

12. The method of claim 1, wherein the bactericidal compound comprises from about 20 to about 40 mM of EDTA and from about 400 to about 600 mM NaNO₂.

13. The method of claim 1, wherein the bactericidal compound comprises about 33 mM of EDTA and about 500 mM NaNO₂.

14. A composition comprising a therapeutically effective amount of acidified NaNO₂ (A-NO₂—) and Na₂-EDTA and a water-based gel.

15. The composition of claim 14, wherein the water-based gel comprises glycerol, an absorbent polymer, and water.

16. The composition of claim 14, wherein the water-based gel comprises glycerol in the range from about 10 to about 30 percent.

17. The composition of claim 14, wherein the bactericidal compound has a pH in the range from about 6.0 to about 6.5.

18. The composition of claim 14, wherein the bactericidal compound comprises from about 1 to about 5 mM of EDTA and from about 20 to about 40 mM of NaNO₂.

19. The composition of claim 14, wherein the bactericidal compound comprises from about 20 to about 40 mM of EDTA and from about 400 to about 600 mM NaNO₂.

20. The composition of claim 14, wherein the bactericidal compound has a ratio of acidified NaNO₂ (A-NO₂—) and Na₂-EDTA to water-based gel of about 1:100.
